SEQUENCE LISTING (82 sequences)

SEQ ID NO: 1 - DNA, 19 bp, Artificial Sequence
  Primer
cgactacatcaccctatcc

SEQ ID NO: 2 - DNA, 16 bp, Artificial Sequence
  Primer
gcaacttcacttgaag

SEQ ID NO: 3 - DNA, 15 bp, Artificial Sequence
  Primer
gcgcaacagaggagc

SEQ ID NO: 4 - DNA, 12 bp, Artificial Sequence
  Primer
ctgaggggatcc

SEQ ID NO: 5 - DNA, 33 bp, Artificial Sequence
  Primer
caaatgtgtatttaagtttagtgaatagttata

SEQ ID NO: 6 - DNA, 24 bp, Artificial Sequence
  Primer
cctatcacaattgtcacattgagg

SEQ ID NO: 7 - DNA, 21 bp, Artificial Sequence
  Primer
atggcttactctaaaatcaca

SEQ ID NO: 8 - DNA, 19 bp, Artificial Sequence
  Primer
gtgctttagctgctgttgc

SEQ ID NO: 9 - DNA, 18 bp, Artificial Sequence
  Primer
atggcttacatcaagatc

SEQ ID NO: 10 - DNA, 21 bp, Artificial Sequence
  Primer
ttataataacatggcggcagc

SEQ ID NO: 11 - DNA, 22 bp, Artificial Sequence
  Primer
atggcttactctaaaatcacat

SEQ ID NO: 12 - DNA, 21 bp, Artificial Sequence
  Primer
tcatagtagcatggcggcaac

SEQ ID NO: 13 - DNA, 22 bp, Artificial Sequence
  Primer
atggttaatataagcatcgtag

SEQ ID NO: 14 - DNA, 19 bp, Artificial Sequence
  Primer
ctatcgttgctgttgtcgc

SEQ ID NO: 15 - DNA, 13 bp, Artificial Sequence
  Primer
atggagggccgca

SEQ ID NO: 16 - DNA, 22 bp, Artificial Sequence
  Primer
cacagaatactttttttgttga

SEQ ID NO: 17 - DNA, 25 bp, Artificial Sequence
  Primer
tgactaaatctggaaaaccacatgg

SEQ ID NO: 18 - DNA, 24 bp, Artificial Sequence
  Primer
caaatatgccaacattgttgacag

SEQ ID NO: 19 - DNA, 22 bp, Artificial Sequence
  Primer
atcgataggcactgcacagtgg

SEQ ID NO: 20 - DNA, 22 bp, Artificial Sequence
  Primer
atgacactgtttggggtttgcc

SEQ ID NO: 21 - DNA, 22 bp, Artificial Sequence
  Primer
atcgataggcactgcacagtgg

SEQ ID NO: 22 - DNA, 21 bp, Artificial Sequence
  Primer
atgacactgtttggggtttgc

SEQ ID NO: 23 - DNA, 28 bp, Artificial Sequence
  Primer
gcaagcttatgacaatgcctcatcgcta

SEQ ID NO: 24 - DNA, 31 bp, Artificial Sequence
  Primer
ggatcccaggaataacgaccgttaataaatt

SEQ ID NO: 25 - DNA, 22 bp, Artificial Sequence
  Primer
gttttattctatatcggtagag

SEQ ID NO: 26 - DNA, 32 bp, Artificial Sequence
  Primer
ggcgggccggactatatttatatatttattaa

SEQ ID NO: 27 - DNA, 26 bp, Artificial Sequence
  Primer
ggatatgtctgggctattttaacagc

SEQ ID NO: 28 - DNA, 33 bp, Artificial Sequence
  Primer
tcctaataaaccaattgttgagatacctgctcc

SEQ ID NO: 29 - DNA, 20 bp, Artificial Sequence
  Primer
gttcaccggattggtcccgc

SEQ ID NO: 30 - DNA, 23 bp, Artificial Sequence
  Primer
gttaattgtaatcttaataaatc

SEQ ID NO: 31 - DNA, 31 bp, Artificial Sequence
  Primer
cagctggacatcatgttggatatgaaacaac

SEQ ID NO: 32 - DNA, 26 bp, Artificial Sequence
  Primer
ggatatgtctgggctattttaacagc

SEQ ID NO: 33 - DNA, 30 bp, Artificial Sequence
  Primer
gtctaatctcaaattgatctaatggtgatg

SEQ ID NO: 34 - DNA, 22 bp, Artificial Sequence
  Primer
atggcatttagaaaatcaaatg

SEQ ID NO: 35 - DNA, 20 bp, Artificial Sequence
  Primer
agctatttttagtggtatgg

SEQ ID NO: 36 - DNA, 20 bp, Artificial Sequence
  Primer
tttatttacagttcatcctg

SEQ ID NO: 37 - DNA, 2815 bp, Saccharomyces pastorianus
agctatttttagtggtatggcaggaacagcaatgtctttaatcattagattagaattagctgcacctggttcacaatatttacaaggaaatgctcagttatttaatgttttagtagttggtcatgctgtattaatgattttctgtgcgccattttgcttaatttatcactgtattgaagtgttaattgataaacatatctctgtttattcaataaatgaaaactttaccgtatcattttggttctgattcttagtagtaacatacatagaatttagatacgtaaaccatatggcttactcagttggggccaactcaacggggacaatagcatgccataaaagcgctggagtaaaacagccagtgcaaggtaagaactgttcgatggctaggttaacgaactccttacaagaatgtttagggttctcattaactccttcccactcggggattgtggttcatgcttgtgtattggaagaagaggtacacgagttaaccaaatatgaatcattaactttaagtaaaagttgacattcggagagctgtacgagttcaaatggtaaattaagaaatatgggattgtccgaaaggggaaactctggggataacggagtcttcatagtacccaaatttaatttaaataaagtgagatattttagtactttatctagattaaatgtaaggaaggaagacagtttaacgtatttaacaaagataaatactacggatttttccgagttaaataaattaatagaaaataattataataatcctgaaaacattaatactagaattttaaaattaatgtcagatattagattgttattaattgcttataataaaattaaaagtaagaaaggtaacatatctaaaggttctaataatattaccttagatggaattaatatttcatatttaaataaattatctaaagatattaatactaatatgtttaaattttctcctgttagaagagttgaaattcctaaaacatctggaggatttagacctttaagtgttggtaatcctagagaaaaaattgtacaagaaagtatgagaataattttagaaattatttataataatagtttttctaattattcacatgggtttagaccaaacttatcttgtttaacagctattattcattgtaaaaattatatgcaacactgtaattgatttattaaggtagacttaaataaatgttttgatacaattccacataatatgttaattaatgtattaaatgagagaatcaaagataaaggtttcattgatttattatataaattattaagagctggatatgttgataaacataataattatcatcatacaactttaggaattcttcaaggtagtgttgtcagtcctattttatgtaatattttcttagataaattagataaatatttagaaaataagtttgagaatgaattcaatattggatctatgtctaatagaagtagaaatccaatttataatgatttatcatctaaaattagaagatgtaaattattatctgataaattaaaattgattagattaagagaccattaccaaagaaatttgggatctgataaaagctttaaaagagcttattttgttagatatgctgatgatattatcattggtgtaatgggttctcatgatgattgtaaaaatattttaaacgatataaataatttcttaacagaaaatttaggtatgtctattaatatagataaatccattattaaacattctaaagaaggagttagttttttagggtatgatgtaaaagttacaccttgagaaataagaccttatagaatgattaaaaaaggtgataaatttattagggttagacatcatactagtttagttgttaatgcccctatcagaagtattgtaataaaattaaataaaaatggttattgttctcatggaatagttggaaaacccataggggttggaagattaattcatgaagaaatgaaaaccattttaatgcattatttagctgttggtagaggtattataaattattatagattagctaccaattttactacattaagaggtagaattacatacattttattttattcatgttgtttaacgttagcaagaaaatttaaattaaatactgttaagaaagttattttaaaattcggtaaagtattgaccgatcctaattcaaaagtaagttttggtattgatgattttaaaattagacataaaataaataaaactgattctaattatactcctgatgaaattttagatagatttaaatatatgttacctagatctttatcattatttagtggtatttgtcaagtttgtggttctaaacaaaatttagaagtacatcatgtgaaaatattaaataatgctgccaataaaatcaaaaatgattatttattaggtagaatgattaagataaatagaaaacaaattactatctgtaaaacatgtcattataaaattcatcaaggtaaatataatggtccaggtttataataattattttaactattaactacgcgttaaatggagagccgtatgatatgaaagtatcacgtacggttcggagagggctctcttatatgattgttaatacaatcagataggtttgctactctactcttagtaatgccagctttaattgggggttttggtaattatttattacccttaatgattggtgctacagatacagcatttccaagaattaataatattgcattttgagtattacctatgggattagtatgtttagttacatcaactttagtagaatcaggtgctggtacaggatgaactgtaaataaa

SEQ ID NO: 38 - DNA, 20 bp, Artificial Sequence
  Primer
ctacagatacagcatttcca

SEQ ID NO: 39 - DNA, 20 bp, Artificial Sequence
  Primer
cgctgtaatgaaaattgatc

SEQ ID NO: 40 - DNA, 985 bp, Saccharomyces pastorianus
ctacagatacagcatttccaagaattaataatattgcattttgagtattacctatgggattagtatgtttagttacatcaactttagtagaatcaggtgctggtacaggatgaactgtaaataaaaaggatatgcagttttaaaatatcatttaatgcataaaataccttatatataataaatattatatataattattaaacatacttaatatatatatatatattaataataatatattaagttataatgtttaataatataggttaatatgcaaatatttataatattaataaatatttcagagactaaatatgatataatatattaatatattaattatctttttataaaaaaaaactattctcattgtaccgaccgttagatacgacgatcgacactattaaatatgatatttataattaataattattttctttataaaatcaatttgatgaataatataattagatttaattacatttatgaaatatttataaatataatttaattaatatataattttttcccgtggatcaaccctattaacaactgggttgtaatttgggggtaataaatattattatattatttttttattataaaaataaagtatataaatactttatattactataataatttttttatatatttataatataattaatttatattaatatattaaagacatagtccgaacaatatagtaatatattgagatatagatattatatatatatttatataaacaattataataattaaatattatttaattattaatttatgatatccaccattatcatctattcaggcacattcaggacctagtgtagatttagcaatttttgcattacatttaacatcaatttcatcattattaggtgctattaatttcattgtaacaacattaaatatgagaacaaatggtatgacaatgcataaattaccattatttgtatgatcaattttcattacagcg

SEQ ID NO: 41 - DNA, 29 bp, Artificial Sequence
  Primer
aatggttattatgcatatgatggcattac

SEQ ID NO: 42 - DNA, 25 bp, Artificial Sequence
  Primer
cctgtaatacctaatggattagatg

SEQ ID NO: 43 - DNA, 24 bp, Artificial Sequence
  Primer
caggatgacctaaagtatttggtg

SEQ ID NO: 44 - DNA, 66 bp, Saccharomyces pastorianus
aatggttattatgcatatgatggcattacatattcatggttcatctaatccattaggtattacagg

SEQ ID NO: 45 - DNA, 188 bp, Saccharomyces pastorianus
aatggttattatgcatatgatggcattacatattcatggttcatctaatccattaggtattacaggtaatttagatagaattccaatgcattcatatttcgtatttaaagatttagtaactgtatttttatttatgttagtattagcattatttgtattttattcaccaaatactttaggtcatcctg

SEQ ID NO: 46 - DNA, 18 bp, Artificial Sequence
  Primer
cgctcctcgtgctgtctt

SEQ ID NO: 47 - DNA, 20 bp, Artificial Sequence
  Primer
ttgacccataccgaccatga

SEQ ID NO: 48 - DNA, 23 bp, Artificial Sequence
  Primer
gtaacagctgcagatgttattca

SEQ ID NO: 49 - DNA, 23 bp, Artificial Sequence
  Primer
ccatagaaaacaccttctctttg

SEQ ID NO: 50 - DNA, 1605 bp, Saccharomyces cerevisiae
atggtacaaagatgattatattcaacaaatgcaaaagatattgcagtattatattttatgttagctatttttagtggtatggcaggaacagcaatgtctttaatcattagattagaattagctgcacctggttcacaatatttacatggtaattcacaattatttaatgttttagtagttggtcatgctgtattaatgattttcttcttagtaatgcctgctttaattggaggttttggtaactatttattaccattaataattggagctacagatacagcatttccaagaattaataacattgctttttgagtattacctatggggttagtatgtttagttacatcaactttagtagaatcaggtgctggtacagggtgaactgtctatccaccattatcatctattcaggcacattcaggacctagtgtagatttagcaatttttgcattacatttaacatcaatttcatcattattaggtgctattaatttcattgtaacaacattaaatatgagaacaaatggtatgacaatgcataaattaccattatttgtatgatcaattttcattacagcgttcttattattattatcattacctgtattatctgctggtattacaatgttattattagatagaaacttcaatacttcattctttgaagtatcaggaggtggtgacccaatcttatacgagcatttattttgattctttggtcaccctgaagtatatattttaattattcctggatttggtattatttcacatgtagtatcaacatattctaaaaaacctgtatttggtgaaatttcaatggtatatgctatggcttcaattggattattaggattcttagtatgatcacatcatatgtatattgtaggattagatgcagatcttagagcatatttcctatctgcactaatgattattgcaattccaacaggaattaaaattttctcatgattagctctaatccatggtggttcaattagattagcactacctatgttatatgcaattgcattcttattcttattcacaatgggtggtttaactggtgttgccttagctaacgcctcattagatgtagcattccacgatacttactacgtggtgggacattttcactatgtattatcaatgggtgctattttctctttatttgcaggatactattattgaagtcctcaaattttaggtttaaactataatgaaaaattagctcaaattcaattctgattaattttcattggggctaatgttattttcttcccaatgcattttttaggtattaatggtatgcctagaagaattcctgattatcctgatgctttcgcaggatgaaattatgtcgcttctattggttcattcattgcactattatcattattcttatttatctatattttatatgatcaattagttaatggattaaacaataaagttaataataaatcagttatttataataaagcacctgattttgtagaatctaatcttatctttaatttaaatacagttaaatcttcatctatcgaattcttattaacttctccaccagctgtacactcatttaatacaccagctgtacaatcttaa

SEQ ID NO: 51 - DNA, 1605 bp, Saccharomyces pastorianus
atggtacaaagatgattatattcaacaaatgcaaaagatattgcagtattatattttatgttagctatttttagtggtatggcaggaacagcaatgtctttaatcattagattagaattagctgcacctggttcacaatatttacaaggaaatgctcagttatttaatgttttagtagttggtcatgctgtattaatgattttcttcttagtaatgccagctttaattgggggttttggtaattatttattacccttaatgattggtgctacagatacagcatttccaagaattaataatattgcattttgagtattacctatgggattagtatgtttagttacatcaactttagtagaatcaggtgctggtacaggatgaactgtatatccaccattatcatctattcaggcacattcaggacctagtgtagatttagcaatttttgcattacatttaacatcaatttcatcattattaggtgctattaatttcattgtaacaacattaaatatgagaacaaatggtatgacaatgcataaattaccattatttgtatgatcaattttcattacagcgttcttattattattatcattacctgtattatctgcaggtattacaatgttattattagatagaaacttcaatacttcattctttgaagtagcaggtggtggtgacccaatcttatatgaacatttattttgattttttggtcatccagaagtatatattttaattattcctggatttggtattatttcacatgtagtatcaacatattctaaaaaaccagtatttggtgaaatttcaatggtatatgctatggcatctattggattattaggattcttagtatgatcacatcatatgtatattgtaggattagatgcagatactagagcctattttctttccgcaactatgatcattgctattcctacaggaattaaaatcttttcatgattagcaacggtctatggtggttcaattagattagcactacctatgttatatgcaattgcattcttattcttattcactatgggtggtttaactggtgttgccttagctaacgcctcattagatgtagcattccacgatacttattacgtagtgggacattttcattatgtattatcaatgggtgctattttctcattatttgcaggatactattattgaagtcctcaaattttaggtttaaattataacgaaaaattagctcaaattcaattctgattaatcttcatcggggctaatgttatcttcttccctatgcatttcttaggtattaatggtatgcccagaagaattccagattaccctgatgctttcgctggttgaaattatgtagcttctattggttcatttattgcacttatctcattattcttattcatctatattttatatgatcaattagttaatggtttaaataataaagttactaataaatcagttgtttactctaaagcacccgattttgttgaatctaatatgatcttcaatttaaatacagttaaatcttcatctattgaattcttattaacttctccaccagctgtacattcattcaatacaccagctgtacaatcttaa

SEQ ID NO: 52 - protein, 534 aa, Saccharomyces cerevisiae
MVQRWLYSTNAKDIAVLYFMLAIFSGMAGTAMSLIIRLELAAPGSQYLHGNSQLFNVLVVGHAVLMIFFLVMPALIGGFGNYLLPLMIGATDTAFPRINNIAFWVLPMGLVCLVTSTLVESGAGTGWTVYPPLSSIQAHSGPSVDLAIFALHLTSISSLLGAINFIVTTLNMRTNGMTMHKLPLFVWSIFITAFLLLLSLPVLSAGITMLLLDRNFNTSFFEVSGGGDPILYEHLFWFFGHPEVYILIIPGFGIISHVVSTYSKKPVFGEISMVYAMASIGLLGFLVWSHHMYIVGLDADTRAYFTSATMIIAIPTGIKIFSWLATIHGGSIRLATPMLYAIAFLFLFTMGGLTGVALANASLDVAFHDTYYVVGHFHYVLSMGAIFSLFAGYYYWSPQILGLNYNEKLAQIQFWLIFIGANVIFFPMHFLGINGMPRRIPDYPDAFAGWNYVASIGSFIATLSLFLFIYILYDQLVNGLNNKVNNKSVIYNKAPDFVESNTIFNLNTVKSSSIEFLLTSPPAVHSFNTPAVQS

SEQ ID NO: 53 - protein, 534 aa, Saccharomyces pastorianus
MVQRWLYSTNAKDIAVLYFMLAIFSGMAGTAMSLIIRLELAAPGSQYLQGNAQLFNVLVVGHAVLMIFFLVMPALIGGFGNYLLPLMIGATDTAFPRINNIAFWVLPMGLVCLVTSTLVESGAGTGWTVYPPLSSIQAHSGPSVDLAIFALHLTSISSLLGAINFIVTTLNMRTNGMTMHKLPLFVWSIFITAFLLLLSLPVLSAGITMLLLDRNFNTSFFEVAGGGDPILYEHLFWFFGHPEVYILIIPGFGIISHVVSTYSKKPVFGEISMVYAMASIGLLGFLVWSHHMYIVGLDADTRAYFTSATMIIAIPTGIKIFSWLATVYGGSIRLATPMLYAIAFLFLFTMGGLTGVALANASLDVAFHDTYYVVGHFHYVLSMGAIFSLFAGYYYWSPQILGLNYNEKLAQIQFWLIFIGANVIFFPMHFLGINGMPRRIPDYPDAFAGWNYVASIGSFIATISLFLFIYILYDQLVNGLNNKVTNKSVVYSKAPDFVESNMIFNLNTVKSSSIEFLLTSPPAVHSFNTPAVQS

SEQ ID NO: 54 - DNA, 1158 bp, Saccharomyces cerevisiae
atggcatttagaaaatcaaatgtgtatttaagtttagtgaatagttatattattgattcaccacaaccatcatcaattaattattgatgaaatatgggttcattattaggtttatgtttagttattcaaattgtaacaggtatttttatggctatgcattattcatctaatattgaattagctttttcatctgttgaacatattataagagatgtgcataatggttatattttaagatatttacatgcaaatggtgcatcattcttttttatggtaatgtttatgcatatggctaaaggtttatattatggttcatatagatcaccaagagtactattatgaaatgtaggtgttattattttcattttaactattgctacagcttttttaggttattgttgtgtttatggacagatgtcacattgaggtgcactagttattactaatttattctcagcaattccatttgtaggtaacgatattgtatcttgattatgaggtgggttctcagtatctaaccctctaatccagagattctttgcgttacattatttagtaccttttatcattgctgcaatggttattatgcatttaatggcattacatattcatggttcatctaatccattaggtattacaggtaatttagatagaattccaatgcattcatactttatttttaaagatttagtaactgttttcttatttatgttaattttagcattatttgtattctattcacctaatactttaggtcatcctgataactatattcctggtaatcctttagtaacaccagcatctattgtacctgaatgatacttattaccattctatgctattttaagatctattcctgataaattattaggagttattctaatgtttgcagctattttagtattattagttttaccatttactgatagaagtgtagtaagaggtaatacttttaaagtattatctaaattcttcttctttatctttgtattcaatttcgtattattaggacaaattggagcatgccatgtagaagtaccttatgtcttaatgggacaaatcgctacatttatctacttcgcttatttcttaattattgtacctgttatctctactattgaaaatgttttattctatatcggtagagttaataaataa

SEQ ID NO: 55 - DNA, 1158 bp, Saccharomyces pastorianus
atggcatttagaaaatcaaatgtatatttaagtttagtgaacagttatgttattgattcaccacaaccatcatcaattagttattgatgaaatataggttcattattaggtttatgtttagttattcaaattttaacaggtatttttatggctatgcattattcatctaatattgagttagcattttcatcagtagaacatattatgagagatactcataatggttatattttaagatatttacatgctaatggtgcatcattcttctttttagttatgtatatgcatatggctaaaggtatgtattatggttcatatagatctccaagagtactattatgaaatgtaggtgttatcattttcgtattaacaattgctacagctttcttaggttattgttgtgtttatgggcagatatcacattgaggtgcactagttattactaatttattttcagcaattccatttgtaggtaatgacattgtagcttgattatgaggaggattctctgtatcaaatcctctaatccaaagattctttgcattacattacttagtaccttttgttattgcagcaatggttattatgcatatgatggcattacatattcatggttcatctaatccattaggtattacaggtaatttagatagaattccaatgcattcatatttcgtatttaaagatttagtaactgtatttttatttatgttagtattagcattatttgtattttattcaccaaatactttaggtcatcctgataattatattccaggtaatcctttagtaacaccagcgtctatcgtacctgaatgatacttattaccattctatgctattttaagatctattcctgataaattattaggtgttattcttatgtttgcagctattttagtgttattagttttaccatttactgatagaagtgttgttagaggtaatactttcaaagtattctctaaattcttcttctttatctttgtattcaatttcgtattattaggacaaattggagcatgtcatgtagaagtaccttatgtcttaatgggacaaattgctacatttatctacttcgcatatttcttaattattgtacctgttatctctactattgaaaatgttttattctatatcggtagagttaataaataa

SEQ ID NO: 56 - protein, 385 aa, Saccharomyces cerevisiae
MAFRKSNVYLSLVNSYIIDSPQPSSINYWWNMGSLLGLCLVIQIVTGIFMAMHYSSNIELAFSSVEHIMRDVHNGYILRYLHANGASFFFMVMFMHMAKGLYYGSYRSPRVTLWNVGVIIFILTIATAFLGYCCVYGQMSHWGATVITNLFSAIPFVGNDIVSWLWGGFSVSNPTIQRFFALHYLVPFIIAAMVIMHLMALHIHGSSNPLGITGNLDRIPMHSYFIFKDLVTVFLFMLILALFVFYSPNTLGHPDNYIPGNPLVTPASIVPEWYLLPFYAILRSIPDKLLGVITMFAAILVLLVLPFTDRSVVRGNTFKVLSKFFFFIFVFNFVLLGQIGACHVEVPYVLMGQIATFIYFAYFLIIVPVISTIENVLFYIGRVNK

SEQ ID NO: 57 - protein, 385 aa, Saccharomyces pastorianus
MAFRKSNVYLSLVNSYVIDSPQPSSISYWWNMGSLLGLCLVIQILTGIFMAMHYSSNIELAFSSVEHIMRDTHNGYILRYLHANGASFFFLVMYMHMAKGMYYGSYRSPRVTLWNVGVIIFVLTIATAFLGYCCVYGQMSHWGATVITNLFSAIPFVGNDIVAWLWGGFSVSNPTIQRFFALHYLVPFVIAAMVIMHMMALHIHGSSNPLGITGNLDRIPMHSYFVFKDLVTVFLFMLVLALFVFYSPNTLGHPDNYIPGNPLVTPASIVPEWYLLPFYAILRSIPDKLLGVITMFAAILVLLVLPFTDRSVVRGNTFKVFSKFFFFIFVFNFVLLGQIGACHVEVPYVLMGQIATFIYFAYFLIIVPVISTIENVLFYIGRVNK

SEQ ID NO: 58 - DNA, 103 bp, Saccharomyces pastorianus
tatatattatctatattagtttatattataattaattgtcatagtagataagctaggttgataataaataaatattgaatacatattaaatatgaagttgttt

SEQ ID NO: 59 - DNA, 33 bp, Saccharomyces pastorianus
aatactagcatgtgtagattagtcagagatatt

SEQ ID NO: 60 - DNA, 42 bp, Saccharomyces pastorianus
aatatgccgattaaaatcgaagcagtatcattacccaaattt

SEQ ID NO: 61 - DNA, 103 bp, Saccharomyces pastorianus
tatatattatctatattagtttatattataattaattgtcatagtagataagctaagttgataataaataaatattgaatacatattaaatatgaagttgttt

SEQ ID NO: 62 - DNA, 33 bp, Saccharomyces pastorianus
aatactaaaatttaaatattattcatatatatt

SEQ ID NO: 63 - DNA, 42 bp, Saccharomyces pastorianus
aatatgccgattaaaattgaggccgtatcattacccaaattt

SEQ ID NO: 64 - DNA, 103 bp, Saccharomyces cerevisiae; identical to SEQ ID NO: 61

SEQ ID NO: 65 - DNA, 33 bp, Saccharomyces cerevisiae; identical to SEQ ID NO: 62

SEQ ID NO: 66 - DNA, 42 bp, Saccharomyces cerevisiae; identical to SEQ ID NO: 63

SEQ ID NO: 67 - DNA, 38 bp, Saccharomyces cerevisiae
aatatttagccccaattaaattaaattaaattaaattg

SEQ ID NO: 68 - DNA, 110 bp, Saccharomyces cerevisiae
ggttaatggttaatggttaatggttaatggttaatggttaatggttaatggttaatggttaatggttaatggttaatggttaatggttaatggttaatgggggggggcgg

SEQ ID NO: 69 - DNA, 43 bp, Saccharomyces pastorianus
aatatttagccccaattaaattaaattaaattaaattaaattg

SEQ ID NO: 70 - DNA, 103 bp, Saccharomyces pastorianus
ggttaatggttaatggttaatggttaatggttaatggttaatggttaatggttaatggttaatggttaatggttaatggttaatggttaatgggggggggcgg

SEQ ID NO: 71 - DNA, 43 bp, Saccharomyces pastorianus; identical to SEQ ID NO: 69

SEQ ID NO: 72 - DNA, 103 bp, Saccharomyces pastorianus; identical to SEQ ID NO: 70

SEQ ID NO: 73 - DNA, 585 bp, Saccharomyces bayanus
aatattatgttttatttatttgttattttaggtttagtatcttgaatgttatatactattgttagaacatattctaataatcctattgcatataaatatattaaacatggacaaactattgaagttatttgaactattttccctgctgtagttttattaattattgctttcccatcatttattttattatatttatgtgatgaagttatttcaccagctatgactattaaagccattggatatcaatgatattgaaaatatgaatattctgattttattaatgatagtggtgaaacagtagaatttgaatcatatgttattcctgatgatttattagaagaaggtcaattaagattattagatacagatacttcaattgtagtaccagtagatacacatattagatttgtagtaacagctgcagatgttattcatgattttgctattcctagtttaggtattaaagtggacgctactcctggaagattaaaccaagtttccgcattaattcaaagagaaggtgttttctatggatcttgttctgaattgtgtggaactggccatgctaatatgccgattaaaatt

SEQ ID NO: 74 - DNA, 585 bp, Saccharomyces uvarum; identical to SEQ ID NO: 73

SEQ ID NO: 75 - DNA, 585 bp, Saccharomyces cerevisiae
aatattatgttttatttattagttattttaggtttagtatcttgaatgttatatacaattgttataacatattcaaaaaatcctattgcatataaatatattaaacatggacaaactattgaagttatttgaacaatttttccagctgtaattttattaattattgctttcccttcatttattttattatatttatgtgatgaagttatttcaccagctataactattaaagctattggatatcaatgatattgaaaatatgaatattcagattttattaatgatagtggtgaaactgttgaatttgaatcatatgttattcctgatgaattattagaagaaggtcaattaagattattagatactgatacttctatagttgtacctgtagatacacatattagattcgttgtaacagctgctgatgttattcatgattttgctattccaagtttaggtattaaagttgatgctactcctggtagattaaatcaagtttctgctttaattcaaagagaaggtgtcttctatggagcatgttctgagttgtgtgggacaggtcatgcaaatatgccaattaagatc

SEQ ID NO: 76 - DNA, 585 bp, Saccharomyces bayanus
aatattatgttttatttatttgttattttaggtttagtatcttgaatgttatatacaattgttagaacatattctaataatcctattgcatataaatatattaaacatggacaaacaattgaagttatttgaactatttttccagcagtagttttattaattattgcttttccatcatttattttattatatttatgtgatgaagttatttcaccagctataactattaaagctattggatatcaatgatattgaaaatatgaatattctgattttattaatgatagtggtgaaacagtagaatttgaatcatatgttattcctgatgatttattagaagaaggacaattaagattattagatactgatacttctatcgtagtacctgtagatacacatattagatttgtagtaacagctgcagatgttattcatgattttgctattcctagtttaggtattaaagtggatgctactcctggaagattaaaccaagtttccgcattaattcaaagagaaggtgttttctatggtaattgctcagaattatgtggaactggccatgctaatatgccgattaaaatc

SEQ ID NO: 77 - DNA, 585 bp, Saccharomyces pastorianus; identical to SEQ ID NO: 76

SEQ ID NO: 78 - DNA, 585 bp, Saccharomyces carlsbergensis
aatattatgttttatttatttgttattttaggtttagtatcttgaatgttatatacaattgttagaacatattctaataatcctattgcatataaatatattaaacatggacaaacaattgaagttatttgaactatttttccagcagtagttttattaattattgcttttccatcatttattttattatatttatgtgatgaagttatttcaccagctataactattaaagctattggatatcaatgatattgaaaatatgaatattctgattttattaatgatagtggtgaaacagtagaatttgaatcatatgttattcctgatgatttattagaagaaggacaattaagattattagatactgatacttctatcgtagtacctgtagatacacatattagatttgtagtaacagctgcagatgttattcatgattttgctattcctagtttaggtattaaagtggatgctactcctggaagattaaaccaagtttccgcattaattcaaagagaaggtgttttctatggtaattgctcagaattatgtggaactggccatgctaatatgccgattaaaatt

SEQ ID NO: 79 - DNA, 585 bp, Saccharomyces pastorianus; identical to SEQ ID NO: 78

SEQ ID NO: 80 - DNA, 585 bp, Saccharomyces monacensis; identical to SEQ ID NO: 78

SEQ ID NO: 81 - DNA, 900 bp, Saccharomyces cerevisiae
tataattcataataataatgaatataggagttaattatttttaagttatagttattttattaaatatcattatgtttaatttattaaatacatatattacatcaccattagatcaatttgagattagactattattaggtttacaatcatcatttattgatttaagttgtttaaatttaacaacattttcattatatactattattgtattattagttattttatgtttatatctattaactaataataataataaaattattggttctagatgattaatttcacaagaagctatttatgatactattataaatatggttaaaggacaaatcggaggtaaaaattgaggtttatatttccctatgatctttactttctttatgtttatttttattgctaatttaattagtatgattccttattcattcgctttatcagctcatttagtatttattatttctttaagtattattatttgattaggtaatactattttaggtttatataaacatggatgactattcttctcattatttgttcctactggtacacctttaccattagttcctttattagttattattgaaactttatcttatattgctagatctatttctttaggtttaagattaggttctaatattttagctggtcatttattaatgattattttagctggtttactatttaactttatgttaattaacttatttactttagtattcggttttgtacctttagctataatcttagctattatgatcttagaattcgctatcggtattatccaaagttatgtttgatgtatcttaactgcttcttatttaaaagatgccgtatatttacactaatttatacttataattgtatataaatactttaataatattcttataaatt

SEQ ID NO: 82 - DNA, 840 bp, Saccharomyces cerevisiae
tagttattttattaaatatcattatgtttaatttattaaatacatatattacatcaccattagatcaatttgagattagactattattaggtttacaatcatcatttattgatttaagttgtttaaatttaacaacattttcattatatactattattgtattattagttattttatgtttatatctattaactaataataataataaaattattggttctagatgattaatttcacaagaagctatttatgatactattataaatatggttaaaggacaaatcggaggtaaaaattgaggtttatatttccctatgatctttactttctttatgtttatttttattgctaatttaattagtatgattccttattcattcgctttatcagctcatttagtatttattatttctttaagtattattatttgattaggtaatactattttaggtttatataaacatggatgactattcttctcattatttgttcctactggtacacctttaccattagttcctttattagttattattgaaactttatcttatattgctagatctatttctttaggtttaagattaggttctaatattttagctggtcatttattaatgattattttagctggtttactatttaactttatgttaattaacttatttactttagtattcggttttgtacctttagctataatcttagctattatgatcttagaattcgctatcggtattatccaaagttatgtttgatgtatcttaactgcttcttatttaaaagatgccgtatatttacactaatttatacttataattgtatataaatactttaataata

(19) United States
(12) Patent Application Publication
(10) Pub. No.: US 2011/0294119 A1
(43) Pub. Date: Dec. 1, 2011

(54) NOVEL METHODS OF DIFFERENTIATING YEAST STRAINS AND/OR DETERMINING GENETIC STABILITY OF YEAST STRAINS, AND USES THEREOF

(75) Inventors: Katherine Anne Smart, Nottingham (GB); Tithira Tirangika Wimalasena, Nottingham (GB); Sarah Michelle Nicholls, Nottingham (GB)

(73) Assignee: THE UNIVERSITY OF NOTTINGHAM, Nottingham (GB)

(21) Appl. No.: 13/055,733

(22) PCT Filed: Jul. 29, 2009
(86) PCT No.: PCT/GB09/50928

§ 371 Date: Aug. 3, 2011

(30) Foreign Application Priority Data
Jul. 26, 2008 (GB) 0813722.6

(51) Int. Cl. C12Q 1/68; C07H 21/04

(52) U.S. Cl. 435/6.11; 435/6.12; 536/24.32; 536/24.33; 435/6.15

(57) ABSTRACT

The invention relates to a method of determining the strain or strains of yeast in a sample, comprising: obtaining and screening nucleic acid from yeast for target sequences comprises all or part of a gene, or a flanking region associated with a gene, in the yeast mitochondrial DNA; and determining from the results of the screen the yeast strain or strains in the sample. Also provided is a method of determining the genetic stability of a yeast strain in a sample, wherein one target sequences in the nucleic acid comprises all or part of a gene, or a flanking region associated with a gene, in the yeast mitochondrial DNA or all or part of a gene, or a flanking region associated with a gene, located in the subtelomeric region of a chromosome; and determining from the results of the screen if the yeast strain is genetically stable.

The present invention relates to a method for differentiating yeast strains and to a method for determining the genetic stability of a yeast strain, and to the use of these methods and to kits for performing these methods.

Identification of yeasts is important in several fields, ranging from the identification of clinical infections and food contaminants, to the control of yeasts used commercially in the production of beer, wine, distilled products, alcohol-based biofuels and food, for example in bread making, for soy sauce production or in the production of natural supplements and probiotics. Each of these applications requires one or more specific types of yeast and the selection and control of the yeast used in each process is of critical importance for product quality and consistency.

The identity of the yeast or yeasts used in a particular process is critical to the quality of the product produced by the process, and manufacturers go to great lengths to control the yeast used. For example, in the brewing industry, companies use proprietary yeast strains that give each of their products characteristics specific to a particular brand. It is therefore crucial that these proprietary yeast strains are maintained and used in pure form, free from contamination.

In order to maintain the integrity of a yeast, master yeast samples are usually stored in facilities known as banks under highly controlled conditions. This provides a standard source of yeast in case the batches of yeast actually used prove to be contaminated. The yeast to be used in a fermentation is usually obtained from propagation of a small sample, which is then either used fresh or dried for reconstitution and later use.

During propagation it is possible that the yeast may change its characteristics or become contaminated. Although growth conditions in the propagator are highly controlled, ensuring that any stress that might induce mutation is kept to a minimum and the risks of contamination are small, mutation and contamination cannot be completely eliminated. Different production methods are used where either successive samples are taken from propagators and used in fermentation, or batches of propagated yeast are removed from one fermentation, retained and used in a subsequent fermentation. Obviously the chance that the yeast has mutated or become contaminated during the full fermentation process is more likely than during the controlled propagation but both methods have some risk. In both cases it is important to ensure that the yeast used in the fermentation is the right yeast and contains no contaminants.

Identification of yeasts, which often vary by very small degrees, is very difficult and existing methods are too slow to be of assistance in the routine monitoring of fermentations.

Currently the identity of a yeast strain is validated using a number of techniques; however the techniques are time-consuming and cannot be run in-house by most manufacturers which use yeast fermentation, such as a brewery. With particular reference to the brewing industry, it is uncommon for the results of strain validation studies to be known before the brewer starts to use a yeast strain in a fermentation system. Thus there are significant risks that the fermentation may be well underway before any contamination or error is identified. This clearly has significant cost implications. Similar situations exist in other fermentation process such as the production of other alcoholic beverages and also in the production of biofuels.

There is therefore a need for a yeast identification method which would allow the yeast strain to be used in a fermentation system to be rapidly validated prior to use.

According to a first aspect, the invention provides a method of determining the strain or strains of yeast in a sample, comprising:
obtaining nucleic acid from yeast in the sample;
screening the nucleic acid for two or more target sequences, wherein at least one of the target sequences in the nucleic acid comprises all or part of a gene, or a flanking region associated with a gene, in the yeast mitochondrial DNA;
determining from the results of the screen the yeast strain or strains in the sample.

Preferably the screening step of the invention is performed using PCR to amplify the two or more target sequences, if present, in the nucleic acid obtained from the yeast sample. Preferably the PCR is carried out using a primer or a pair of primers directed to each of the target sequences. Preferably a pair of primers is used for each target sequence being screened for. The PCR may be Real-Time PCR. A melt curve may be performed to determine if the correct product has been amplified.

Alternatively, one or more probes may be used to determine the presence or absence of the two or more target sequences. The one or more probes may be labelled.

Preferably the presence or absence of the target sequence in the nucleic acid sample is determined by detecting the presence or absence of an amplification product from the PCR reaction. This may be done by using gel electrophoresis. As well as the presence of an amplification product, the size and/or sequence of the amplification product may be considered when determining whether a particular target sequence is present.

Preferably, in the method of the invention, at least one of the target sequences comprises all or part of at least one non-mitochondrial gene, or the flanking region associated with at least one non-mitochondrial gene.

The non-mitochondrial gene, and/or flanking sequence thereof, may be a gene which encodes a protein selected from the group comprising the yeast cell wall associated protein, proteins associated with sugar metabolism, mitochondrion associated proteins and transcription factors and proteins involved with yeast stress.

In one embodiment of the invention only mitochondrial genes, and/or their flanking regions, need to be used as the target sequences. For example, yeasts used to produce ale can be distinguished from yeasts used to produce lager on the basis of their mitochondrial DNA only. Similarly, many wild yeasts can be distinguished from many commercial yeasts on the basis of target sequences in the mitochondrial DNA only. However, for other yeast strains it is preferable to also use chromosomal genes, and/or their flanking regions, to distinguish between strains. For example, to distinguish between lager yeast strains.

The method of the invention may use sets of PCR primers or probes tailored to different yeast strains. Each set of primers or probes may include primers designed to amplify two or more target sequences in the yeast nucleic acid, wherein at least one of the target sequences is in the yeast mitochondrial DNA. Each set of primers or probes may include probes designed to detect two or more target sequences in the yeast nucleic acid, wherein at least one of the target sequences is in the yeast mitochondrial DNA. Preferably each set of primers or probes includes primers or probes directed to three or more, four or more, five or more, six or more, seven or more, eight or more, nine or more, ten or more etc, target sequences in the yeast nucleic acid.

The method of the invention may exploit differences in the gene order/location between different strains.

The manufacturer performing the yeast fermentation process will usually know what yeast they should be using and what the likely contaminants are, thus the method of the invention can be tailored to screen for these strains. Likely contaminants are usually other strains used in the manufacturing plant and/or wild yeasts.

Examples of wild yeasts, that might occur in the process or product and cause spoilage, include the genera Pichia, Hanseniaspora, Zygosaccharomyces, Candida and Dekkera/Brettanomyces species.

Strains of yeast used in wine production include strains from the species Saccharomyces, and Saccharomyces cerevisiae in particular.

Strains of yeast used in ethanol (biofuel) production include strains from the species Saccharomyces cerevisiae which is generally used for molasses fermentation, and Kluyveromyces cellobiovorus which may be used for cellulose fermentation.

Strains of yeast used in beer production include Saccharomyces cerevisiae, a so called “top fermenting” yeast or ale yeast strain used in the production of ale-type beers (ales).

An ale yeast strain may be selected from any of the group comprising UNYC7 (S. cerevisiae), UNYC8 (S. cerevisiae), UNYC9 (S. cerevisiae), UNYC10 (S. cerevisiae), NCYC 1119 (S. cerevisiae), NCYC 2593 (S. cerevisiae), and KS1 (S. cerevisiae), or combinations thereof.

Lager yeast strains include Saccharomyces carlsbergensis, Saccharomyces pastorianus, Saccharomyces uvarum, W34 from Weihenstephan in Germany or a hybrid of Saccharomyces cerevisiae and Saccharomyces bayanus, all of which are so called “bottom fermenting” yeasts used in the production of lager-type beers (lagers).

A lager yeast strain may also be selected from any of the group comprising UNYC3 (S. cerevisiae (sy. Pastorianus)), UNYC4 (S. cerevisiae (sy. Pastorianus)), UNYC5 (S. cerevisiae (sy. pastorianus)), UNYC6 (S. cerevisiae (sy. Pastorianus)), UNYC2 (S. cerevisiae), UNYC1 (S. carlsbergensis) and NCYC1116 (S. carlsbergensis) or combinations thereof.

Ale producing yeast strains are generally more stable and easier to distinguish than lager producing strains. This may be due to the fact that ale producing strains are older, in evolutionary terms.

Preferably the method of the invention allows two, three, four, five, six, seven, eight, nine, ten or more strains of yeast to be identified in one sample.

Traditional methods for differentiating, identifying and characterising yeasts, and in particular brewing yeasts, are based on biochemical, morphological and physiological criteria of growing yeast and on fermentation characteristics such as flocculation. Such methods are time-consuming (taking several days to a week) to perform and often provide inconclusive or give incomplete results. For example, currently, in order to determine the strain of yeast present in a particular beer fermentation process, brewers will take a sample of a brewing beer (lager or ale), or the propagation culture used for a particular fermentation, and give it to a laboratory who will then grow cultures of the yeast in the sample and then perform a series of “yes/no” tests to determine certain phenotypes of the yeast. From the results of the “yes/no” tests the strain of yeast may be determined.

Although molecular techniques have recently been used to differentiate the strains of yeast in a sample of beer, these techniques use the differences in ploidy, chromosome length polymorphisms or sequence differences between yeast strains to distinguish different yeast strains (Smart K. A. (2007) Brewing yeast genomes and genome-wide expression and proteome profiling during fermentation. Yeast. Epub ahead of print). Typically, the molecular techniques used include restriction fragment length polymorphism (RFLP) (Hammond, J. (2002) Yeast Genetics In Brewing Microbiology. Kluwer Academic: Dordrecht, The Netherlands; 67-113; Meaden, P. (1990) J. Inst. Brew. 96: 195-200), gene specific probes targeting HIS4 and LEU2 (Pedersen, M. B. (1985) Carlsb. Res. Commun. 50: 263-272) and pulse field gel electrophoresis (PFGE) (Casey, G. P., Pringle, A. T., and Erdmann, P. A. (1990) J. Am. Soc. Brew. Chem. 48:100-106). All these techniques need specialist detailed knowledge of molecular methods, and thus will often be outsourced to a third party provider at considerable cost. Furthermore, these methods are all time consuming, and due to the time taken to obtain the results the data provided is retrospective, that is, it is too late to stop and restart a fermentation, such as in a brewing process, if there is a problem with the yeast strain.

The present invention provides a rapid and accurate method to determine the strain or strains of yeast in a sample. Preferably the method can be carried out in less than about 24 hours, preferably less than about 18 hours, preferably less than about 12 hours. The method can preferably be carried out without the need for highly trained technicians and expensive laboratory equipment. The method may be useful in many applications. For example, in the brewing industry it is necessary to maintain the same strain or strains of brewing yeast during the fermentation procedure in order to ensure final product quality. If the yeast strain, or the balance of yeast strains, changes during the fermentation process, then the beer produced may be of reduced quality. It is therefore important to be able to rapidly determine the yeast present at any stage during fermentation.

The present invention provides a versatile customised strain detection method which can provide a robust quality assurance of the yeast strains used in yeast fermentation industries, such as brewing. For example, the present invention can provide unambiguous and reproducible differentiation of proprietary brewing yeast.

The financial importance of the invention can be exemplified in the brewing industry. Beer is typically brewed over four or five days in batches of several thousand litres, often in large “fermentation vessels”. Any one particular vessel may represent an expensive capital investment of over £100,000, emphasising the financial importance of ensuring that the correct strain of yeast is used. Furthermore, a brewery may have a finite production capacity and may only be able to brew a limited number, i.e. 50 or so, of batches of beer per year. Thus, if one of the “pitches”/batches of beer had the wrong yeast in it, and the resulting beer was not of premium quality, then there would be a large financial loss, and the production capacity of the brewery would be effectively limited. An advantage of the present invention is that it allows the yeast in a particular batch to be quickly identified at any stage of the production process, for example after one day, or even after a few hours. If necessary the fermentation can then be stopped and discarded, or stopped and a yeast of the correct strain added.

The method of the first aspect of the invention may be performed on a sample obtained during a fermentation process, such as that used to produce beer.

Alternatively, the method of the invention may be used before a fermentation is started to provide strain verification data before the yeast is used, or supplied to a manufacturing site, for propagation and fermentation.

The method may also be performed on a sample of yeast after propagation but before it is used to inoculate a fermentation reaction.

The method of the invention may be used to ensure the quality of yeast used in any fermentation process, the yeast may be tested at any stage of use. With reference to a product produced by yeast fermentation there are at least three aspects to quality control of yeast during product production; firstly ensuring the right yeast is actually used in the fermentation, secondly testing to ensure lack of contamination with wild or unwanted yeasts and thirdly checking for any genetic drift in the added yeast during propagation or fermentation that might introduce off flavours or inhibit performance.

The method of the invention may be used to identify that the right strain has been selected for initial growth. Since quite basic errors in labelling and supply can occur this is important. At this stage it is necessary to provide a means for positive identification of the desired yeast strain. The method of the invention can be tailored to specific individual strains, by screening for one or more target nucleic acid sequences that are only found in that strain.

The second aspect of quality control is the monitoring of contamination during fermentation. Contamination can occur when wild yeast (that is, any yeast that has not been added in a controlled manner), which can arise from the wide range of sources of yeast in the environment, appears in the fermentation. Usually contaminant yeasts are either other strains used in the fermentation plant that have “escaped” accidentally and entered the fermentation or other spoilage yeasts present in the atmosphere or are introduced by staff. The method of the invention can be tailored to screen for the desired and strain and likely contaminants by using primers directed to target nucleic acid sequences unique to these strains.

The third aspect of quality control is the identification of genetic drift, which can occur during prolonged propagation or during fermentation. Genetic drift can be induced by stress, such as increased alcohol concentration or temperature, which causes changes in genetic sequence which may be very small but critical in terms of flavour of the final product or performance of the yeast. Again, the method of the invention can be designed to screen for target sequences which allow this instability to be seen.

The method of the invention can be used to address all three aspects of quality control.

If, by using the method of the invention during fermentation, an undesirable yeast strain is detected in the sample the fermentation process may be stopped. If appropriate, the fermentation process may then be restarted with the correct strain or strains of yeast. Alternatively, if, by using the method of the invention, the yeast strain identified in the fermentation is not a preferred strain, a decision can be made to use the product being produced in an alternative product. For example, in the case of beer fermentation, if the yeast found in the fermentation process is not the preferred strain, then the beer produced may be used in a different brand, perhaps a less premium brand. If the correct/intended strain or strains of yeast are identified the manufacturer can be confident that the end product will be as expected.

The sample to be used in the method of the invention may be a liquid, slurry or solid. The sample may be taken from a stock culture, from a propagation culture, from a fermentation mix before fermentation begins, from a fermentation mixture during fermentation, or from a fermentation product. The sample may be a culture of cells originally obtained from a fermentation, or a culture of cells intended to be used for fermentation. The nucleic acid may be isolated directly from the sample, or the sample may be cultured before the nucleic acid is isolated.

Nucleic acid for use in any method of the invention may be obtained by extracting nucleic acid from yeast cells in the sample. The nucleic acid may be recovered on site, or it may be extracted from a sample using the services of a third party.

Extracted nucleic acid may comprise whole cell extract, or it may be purified or partially-purified nucleic acid. The nucleic acid used may be total nucleic acid from the yeast, or it may be genomic DNA and/or mitochondrial DNA and/or a mixture thereof.

A target sequence of mitochondrial DNA for use in the method of the invention may comprise at least part of a gene selected from the group comprising COB, COX1, COX2, COX3, ATP8, ATP6, ATP9, VAR1 and RPM1 and/or any non-coding sequences flanking or separating these genes, or combinations thereof.

CYC3, CYC1 and CYC2 are genes involved in encoding mitochondrial peripheral inner membrane proteins.

The target sequence may comprise all or at least part of one or more of the yeast genes selected from the group comprising the TIR genes, the DAN genes, the FLO genes, the ECM genes, the BUD genes, the KEL genes, the MNT genes, the SED genes, the MEL genes, SUC genes, the ATF genes, the GST genes, the GAL genes, MAL1-4, CYC1, CYC2, CYC3, CBP1, CBP2, CBP3, CBP4, and CBT1 and the mitochondrial genes COB, COX1, COX2, COX3, ATP8, ATP6, ATP9, VAR1 and RPM1 and/or a flanking region thereof.

The TIR genes may comprise genes selected from the group comprising TIR1, TIR2 and TIR4, or combinations thereof.

The DAN genes may comprise genes selected from the group comprising DAN1, DAN2, DAN3 and DAN4, or combinations thereof.

The TIR and DAN genes encode nine protein cell wall mannoproteins in Saccharomyces cerevisiae which are expressed during anaerobiosis, namely DAN1, DAN2, DAN3, DAN4, TIR1, TIR2, TIR3 and TIR4.

The ECM genes may comprise genes selected from the group comprising ECM1, ECM2, ECM3, ECM4, ECM5, ECM6, ECM7, ECM8, ECM9, ECM10, ECM11, ECM12 ECM13, ECM14, ECM15, ECM16, ECM17, ECM18, ECM19, ECM20, ECM21, ECM22, ECM23, ECM24, ECM24, ECM26, ECM27, ECM28, ECM29, ECM30, ECM31, ECM32, ECM33, and ECM34, or combinations thereof.

The ECM genes encode extracellular matrix proteins.

ECM33(YBR078W) from Saccharomyces cerevisiae encodes a glycosylphosphatidylinositol (GPI)-attached protein. If this gene is disrupted the cells exhibit a temperature-sensitive growth defect and hypersensitivity to oxidative stress. ECM34 (YBR043W) encodes a protein of unknown function, but the gene is located towards the telomere within subtelomeric sequences.

The FLO genes may comprise genes selected from the group comprising FLO1, FLO5, FLO8, FLO9, FLO10, and FLO11 or combinations thereof.

The flo genes encode lectin-like cell surface proteins which aggregate cells into “flocs” by binding to mannose sugar chains on the surface of other cells. This flocculation of cells is calcium dependent and non-sexual, and is stimulated by nutrient limitation. This process is very important to the brewing characteristics of yeast.

The BUD1, BUD2, BUDS etc genes encode proteins involved in bud-site selection and are required for the axial budding of yeast cells.

The KEL1, KEL2 and KEL3 genes encode proteins required for proper cell fusion and cell morphology.

The MNT1-4 genes encode proteins involve in O-glycosylation.

The SED genes include SED1-6. SED1 encodes a stress induced structural GPI-cell wall glycoprotein which associates with translating ribosomes. It also has a putative role in mitochondrial genome maintenance. SED2 encodes a glycosylated integral membrane protein of the endoplasmic reticulum. SED3 encodes a protein involved in O-mannosylation and protein glycosylation. SED4 encodes a protein associated with the integral endoplasmic reticulum membrane. SED5 encodes a protein required for vesicular transport between the endoplasmic reticulum and the golgi complex. SED6 encodes a protein involved in the zymosterol to fecosterol in the ergosterol biosynthetic pathway.

The advantage of using yeast cell wall related sequences in the method of the invention is that the cell wall associated DNA sequences are polymorphic. This polymorphism provides measurable differences between closely related yeast strains and allows strains to be accurately and reproducibly distinguished by using target sequences in these genes.

A target sequence in the nucleic acid associated with yeast metabolism may comprise all or part of any gene, operon, promoter, or flanking region thereof, which relates to the function or maintenance of yeast cell metabolism, or the expression of proteins related to yeast cell metabolism, preferably metabolism of a substrate. The target sequence in the nucleic acid may be at least part of one or more of the yeast genes selected from the group comprising the MEL genes, the SUC genes, ATF1, ATF2, GSY1, GSY2 and GAL1-10 and/or flanking regions thereof.

The MEL genes may comprise any of the genes selected from the group comprising MEL1, MEL2, MEL3, MEL4, MEL5, MEL6, MEL7, MEL8, MEL9 and MEL10, or combinations thereof.

The MEL genes encode secreted alpha-galactosidase required for the catabolic conversion of melibiose to glucose and galactose. Lager yeast strains are all able to utilise melibiose.

The SUC genes may comprise genes selected from the group comprising SUC1, SUC2, SUC3, SUC4, SUC5, and SUC7, or combinations thereof.

The SUC genes encode invertase, a sucrose hydrolyzing enzyme. This enzyme, also known as sucrase, beta-fructofuranisidase or beta-fructosidase, plays an important role in sucrose metabolism. Invertase catalyzes the hydrolysis of the disaccharide sucrose to fructose and glucose and the trisaccharide raffinose to fructose and melibiose. All SUC genes, except SUC2, are located within subtelomeric sequences.

ATF1 and ATF2 are genes encoding proteins involved in lipid and sterol metabolism; which is responsible for the major part of volatile acetate ester production during fermentation.

GSY1 and GSY2 are genes which encode proteins involved in glycogen synthesis.

GAL1-10 are genes encoding proteins involve in galactose metabolism.

Targeting sequences associated with metabolism, especially substrate metabolism, has the advantage that the method focuses on what substrate each strain is capable of metabolising, which directly reflects on what the final product of the yeast fermentation will be. Yeast strains can be differentiated on the basis of what substrates they can utilise.

CYC1, CYC2, CYC3, CBP1, CBP2, CBP3, CBP4, and CBT1 genes encode proteins associated with mitochondrion but not encoded by the mitochondrial genome.

CYC3, CYC1 and CYC2 are genes involved in encoding the mitochondrial peripheral inner membrane.

CBP1, CBP2, CBP3 and CBP4 are genes encoding mitochondrial proteins that interact with the COB mRNA and have a role in its stability and translation.

CBT1 encodes a protein involved in processing the mitochondrial COB protein.

The two or more target sequences may be selected from the group comprising all or part of one or more of the following genes, or flanking regions thereof, COB, RPM1, ATP9, VAR1, COX1, COX2, COX3, ATP6, ATP8, TIR4, MEL1, FLO1, SUC3, and SUC5. Preferably at least one of the target sequences comprises all or part of one or more of the following genes, or flanking regions thereof, COB, RPM1, ATP9, VAR1, COX1, COX2, COX3, ATP6 and ATP8.

In any method of the invention which utilizes PCR to detect the target sequence, one or more oligonucleotide primers may be used. The primers or probes may be complementary or reverse complementary to the target sequence. The one or more primers or probes may be complementary or reverse complementary to part of one or more of genes or flanking regions thereof, selected from the group comprising TIR genes, such as TIR1, TIR2, TIR4; DAN genes, such as DAN1, DAN2, DAN3 and DAN4; ECM genes, such as ECM1, ECM2, ECM3, ECM4, ECM5, ECM6, ECM7, ECM8, ECM9, ECM10, ECM11, ECM12 ECM13, ECM14, ECM15, ECM16, ECM17, ECM18, ECM19, ECM20, ECM21, ECM22, ECM23, ECM24, ECM24, ECM26, ECM27, ECM28, ECM29, ECM30, ECM31, ECM32, ECM33, and ECM34; FLO genes, such as FLO1, FLO5, FLO8, FLO9, FLO10, and FLO11; BUD1; BUD2; BUDS etc; KEL1; KEL2; KEL3; and MNT1-4; or combinations thereof.

The primers or probes may be complementary or reverse complementary to part of the sequence of or flanking region of one or more gene selected from the group comprising MEL genes, such as MEL1, MEL2, MEL5, and MEL6; SUC genes, such as SUC1, SUC2, SUC3, SUC4, SUC5, and SUC7; ATF1; ATF2; GSY1; GSY2; and GAL1-10; or combinations thereof.

The primers or probes may be complementary or reverse complementary to part of the sequence of or flanking region of one or more gene selected from the group comprising CYC1, CYC2, CYC3, CBP1, CBP2, CBP3, CBP4, and CBT1 or combinations thereof.

The primers or probes may be complementary or reverse complementary to part of the sequence of the yeast mitochondrial DNA, such as part of one or more mitochondrial genes, or flanking regions thereof. The mitochondrial gene may be selected from the group comprising COB, COX1, COX2, COX3, ATP8, ATP6, ATP9, VAR1 and RPM1 or combinations thereof.

The method of the invention may use one or more primers or probes complementary or reverse complementary to part of the sequence of the yeast mitochondrial DNA, such as part of one or more mitochondrial genes, or flanking regions thereof, wherein the mitochondrial gene is preferably selected from the group comprising COB, COX1, COX2, COX3, ATP8, ATP6, ATP9, VAR1 and RPM1 together with one or more primers or probes complementary or reverse complementary to part of the sequence of or flanking region of one or more gene selected from the group comprising MEL genes, such as MEL1, MEL2, MEL3, MEL4, MEL5, MEL6, MEL7, MEL8, MEL 9 and MEL10; MAL1-4, SUC genes, such as SUC1, SUC2, SUC3, SUC4, SUC5, and SUC7; ATF1; ATF2; GSY1; GSY2; GAL1-10, TIR genes, such as TIR1, TIR2, TIR4; DAN genes, such as DAN1, DAN2, DAN3 and DAN4; ECM genes, such as ECM1, ECM2, ECM3, ECM4, ECM5, ECM6, ECM7, ECM8, ECM9, ECM10, ECM11, ECM12 ECM13, ECM14, ECM15, ECM16, ECM17, ECM18, ECM19, ECM20, ECM21, ECM22, ECM23, ECM24, ECM24, ECM26, ECM27, ECM28, ECM29, ECM30, ECM31, ECM32, ECM33, and ECM34; FLO genes, such as FLO1, FLO5, FLO8, FLO9, FLO10, and FLO11; BUD1; BUD2; BUD3 etc; KEL1; KEL2; KEL3; MNT1-4; CYC1; CYC2; CYC3; CBP1; CBP2; CBP3; CBP4 and CBT1 or combinations thereof.

Preferably the method of the invention uses one or more primers or probes complementary or reverse complementary to part of the sequence of the yeast mitochondrial COB gene sequence, or flanking regions thereof, together with one or more primers or probes complementary or reverse complementary to part of the sequence of the MEL1, SED1, SUC5, SUC3, FLO11, FLO1, TIR1, TIR2 and TIR4 genes, or flanking regions thereof. Preferably, these primers or probes allow lager and ale yeast strains to be distinguished.

The primers or probes may comprise a sequence selected from the group comprising SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 38, 39, 41, 42 and 43, or combinations thereof, or a sequence with at least 80%, 85%, 90%, 95%, 98% or more sequence identity to a sequence with the sequence of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 38, 39, 41, 42 and 43.

The primers may comprise an primer pair comprising a forward primer of SEQ ID NO: 1 and a reverse primer of SEQ ID NO: 3, or a sequence with at least 80%, 85%, 90%, 95%, 98% or more sequence identity to the sequence of SEQ ID NOs: 1 and/or 3 and produce the same PCR product as primers of SEQ ID NO: 1 and SEQ ID NO: 3. Preferably the primer pair of SEQ ID NO: 1 and SEQ ID NO: 3 are specific for ale yeast strains.

The primers may comprise a lager primer pair comprising a forward primer of SEQ ID NO: 1 and a reverse primer of SEQ ID NO: 4, or a sequence with at least 80%, 85%, 90%, 95%, 98% or more sequence identity to the sequence of SEQ ID NOs: 1 and/or 4 and produce the same PCR product as primers of SEQ ID NO: 1 and SEQ ID NO: 4. Preferably the primer pair of SEQ ID NO: 1 and SEQ ID NO: 3 are specific for lager yeast strains.

The method of the invention may be used with one or more, two or more, three or more, four or more, five or more, six or more, seven or more, eight or more, nine or more, ten or more of the primer pairs of SEQ ID NO: 1 and SEQ ID NO: 2, SEQ ID NO: 1 and SEQ ID NO: 3, SEQ ID NO: 1 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 15 and SEQ ID NO: 16, SEQ ID NO: 17 and SEQ ID NO: 18, SEQ ID NO: 19 and SEQ ID NO: 20, SEQ ID NO: 21 and SEQ ID NO: 22, SEQ ID NO: 23 and SEQ ID NO: 24, SEQ ID NO: 31 and SEQ ID NO: 26, SEQ ID NO: 32 and SEQ ID NO: 28, SEQ ID NO: 29 and SEQ ID NO: 30, SEQ ID NO: 25 and SEQ ID NO: 28, SEQ ID NO: 28 and SEQ ID NO: 29, SEQ ID NO: 27 and SEQ ID NO: 33, SEQ ID NO: 34 and SEQ ID NO: 6, SEQ ID NO: 35 and SEQ ID NO: 36. SEQ ID NO: 38 and SEQ ID NO: 39, SEQ ID NO: 41 and SEQ ID NO: 42, and SEQ ID NO: 41 and SEQ ID NO: 43 or sequences with at least 80%, 85%, 90%, 95%, 98% or more sequence identity to the sequence of the SEQ ID NOs listed and wherein the sequences produce the same PCR product as primers of the specified SEQ ID NOs.

Preferably two or more of the following primer pairs are used SEQ ID NO: 1 and SEQ ID NO: 3, SEQ ID NO: 1 and SEQ ID NO: 4, SEQ ID SEQ ID NO: 5 and SEQ ID NO: 6, SEQ ID NO: 17 and SEQ ID NO: 18, SEQ ID NO: 19 and SEQ ID NO: 20, SEQ ID NO: 21 and SEQ ID NO: 22, SEQ ID NO: 23 and SEQ ID NO: 24, SEQ ID NO: 31 and SEQ ID NO: 26, SEQ ID NO: 32 and SEQ ID NO: 28, SEQ ID NO: 29 and SEQ ID NO: 30, and SEQ ID NO: 25 and SEQ ID NO: 28, SEQ ID NO: 35 and SEQ ID NO: 36. SEQ ID NO: 38 and SEQ ID NO: 39, SEQ ID NO: 41 and SEQ ID NO: 42, and SEQ ID NO: 41 and SEQ ID NO: 43 or sequences with at least 80%, 85%, 90%, 95%, 98% or more sequence identity to the sequence of the SEQ ID NOs listed and wherein the sequences produce the same PCR product as primers of the specified SEQ ID NOs.

The primers may comprise an ale primer pair and a lager primer pair.

The primers used in a method of the invention may comprise a primer set, the primer set may comprise primer pairs directed to the yeast genomic DNA, together with one or more primer pairs directed to mitochondrial DNA. The probes used in a method of the invention may comprise a probe set, the probe set may comprise probes directed to the yeast genomic DNA, together with one or more probes directed to mitochondrial DNA. A mixture of primers and probes may be used.

The primers or probes may be specific for a specific yeast strain or strains.

The primers or probes may comprise a pair of control primers and/or one or more control probes. The control primers may be designed to amplify a target region of DNA which is common to the different yeast strains being tested. The control primers may comprise a forward primer having the sequence of SEQ ID NO: 1 and a reverse primer having the sequence of SEQ ID NO: 2. The one or more control probes may be designed to detect one or more target regions or sequences of DNA which are common to the different yeast strains being tested.

The benefit of the control primers is that they provide assurance that the PCR amplification was successful and that reaction/testing conditions are correct.

The term “flanking region(s)” may refer to regions of DNA up to about 1000, 500, 300, 100, 50, or 25 nucleotides upstream or downstream from a coding region of a gene. Flanking regions may comprise introns between and/or within the genes.

In addition to determining what stain of yeast is present in a sample, it is also important to understand the stability of the yeast. Yeast are known to mutate over successive generations, and to eventually mutate so much that will behave differently, and indeed produce a different product upon fermentation. This can have a dramatic effect on the quality of the product of a fermentation reaction. A yeast is defined as unstable if it has mutated such that a phenotypic change has occurred, typically this occurs when a yeast is under stress and the appearance of cells known as petites (petite colony mutation) occur. These mutants can be identified under the microscope. These mutants are common and are deficient in mitochondrial DNA, and result it reduced performance of the yeast. For example, if petite mutants occur in the fermentation of beer the taste is affected and thus the product has a reduced value. Petite mutants are also slow growing and thus slow down a fermentation reaction.

According to another aspect of the invention there is provided a method of determining the genetic stability of a yeast strain in a sample, comprising:
obtaining nucleic acid from the yeast in the sample;
screening the nucleic acid for two or more target sequences, wherein at least of the one target sequences in the nucleic acid comprises all or part of a gene, or a flanking region associated with a gene, in the yeast mitochondrial DNA or all or part of a gene, or a flanking region associated with a gene, located at subtelomeric regions;
determining from the results of the screen if the yeast strain is genetically stable.

The subtelomeric region refers to a region of a chromosome proximal to the telomere. In Saccharomyces the telomeric region is approximately 350 base pairs long. Preferably a gene in the subtelomeric region is at least about 500 base pairs from the end of the chromosome.

It will be appreciated that many of the preferred features discussed with reference to the first aspect of the invention can be applied to this aspect, in particular with reference to the invention being preferably performed by PCR, when the method may be performed, the nature of the sample, how the nucleic acid is extracted and the ease of performing the invention.

The method to determine the genetic stability of a yeast strain in a sample may comprise the use of Real-Time PCR (RT-PCR) to determine relative mtDNA (mitochondrial DNA) copy number of a gene in the yeast. A relative reduction in, or a low, mtDNA copy number of a gene may indicate an increased likelihood of the yeast becoming, or being unstable. An absence of, or deletion of, a COX2 gene, in mtDNA may indicate a likelihood of an unstable yeast. The mtDNA copy number may be determined relative to a gene known to remain significantly unchanged in copy number, for example ACT1 gene.

The method to determine the genetic stability of a yeast strain in a sample may comprise the use of a melt curve to determine the product properties and observe the hybridisation properties of primers or probes directed to two or more target sequences.

The RT-PCR or melt curve may use one or more primers selected from any of SEQ ID NO: 46, 47, 48 or 49. The RT-PCR may use a primer pair of SEQ ID NO: 48 and 49. The RT-PCR may use a primer pair of SEQ ID NO: 46 and 47 as a control.

An advantage of this method is that the stability of yeast strains can be rapidly and accurately determined. For example, the present invention can provide an unambiguous and reproducible indication that a proprietary brewing yeast has become or is becoming genetically unstable and should no longer be used in the fermentation process. Traditionally brewers test their beer at the end of the brewing process, and if it is sub-standard due to the yeast mutating too much (becoming unstable), then that batch of beer is either discarded or sold more cheaply. Thus, the brewery loses money. The present invention advantageously allows the stability of the yeast to be verified before it is used in a subsequent batch of brewing, or during the brewing process.

Manufacturers using yeast fermentation may attempt to pre-empt the lack of stability of the yeast by changing the yeast regularly, however, this is undesirable because genetically stable yeast may be unnecessarily discarded. The present invention has the benefit that it identifies the yeast as they start to mutate but before the mutation is sufficient to affect the quality of the product. Thus, the yeast is changed only when it is necessary.

Preferably, the method according to this aspect of the invention is predictive and can be used to indicate when a yeast is becoming unstable. In order for a phenotypic change to be seen in yeast due to genome instability all copies of mitochondrial DNA need to be damaged. Yeast typically have 20 mitochondria, all of which must be damaged to see a phenotypic change. By screening regularly using the method of this aspect of the invention, damage can be seen as it begins to occur, and prior to a phenotypic change. Therefore the yeast can be changed before the genomic damage affects the product. Preferably the method of the invention further includes the step of determining the mitochondrial copy number. Together, the results of the screen for target sequences and the mitochondrial copy number can be used to predict whether or not a yeast strain is becoming genetically unstable.

The term “stability” refers to the genetic instability of yeast. The DNA in some regions of the yeast nuclear and/or mitochondrial DNA becomes unstable over time due to deletions, recombinations or inappropriate insertions and can result in undesirable morphological and/or physiological changes in the yeast. Yeast strains can only be used or re-used for a limited period, or for a limited number of successive fermentations, before they become genetically unstable and unsuitable for their purpose. Detection of this instability is economically important, particularly to the brewing industry.

A target sequence of mitochondrial DNA used in this aspect of the invention may comprise at least part of a gene selected from the group comprising COB, COX1, COX2, COX3, ATP8, ATP6, ATP9, VAR1, and RPM1 and/or any non-coding sequences flanking or separating these genes, or combinations thereof.

A target sequence of subtelomeric DNA used in this aspect of the invention may comprise at least a part of a gene selected from the group comprising ECM34, SUC1, SUC3, SUC4, SUC5, SUC7, MAL1, MAL2, MAL3, MAL4, MEL2, MEL3, MEL4, MEL5, MEL6, MEL7, MEL8, MEL9 and MEL10 and/or any non-coding sequences flanking or separating these genes, or combinations thereof.

Preferably the target sequence is in the subtelomeric region of Chromosome I, VI, X or XI.

The method of this aspect of the invention may use one or more primers or probes, complementary or reverse complementary to part of the sequence of the yeast mitochondrial DNA or a telomere region of a yeast chromosome, in a PCR reaction. The primers or probes may be complementary or reverse complementary to part of the sequence of one or more of the following genes COB, COX1, COX2, COX3, ATP8, ATP6, ATP9, VAR1, RPM1, ECM34, SUC1, SUC3, SUC4, SUC5, SUC7, MAL1, MAL2, MAL3, MAL4, MEL2, MEL3, MEL4, MEL5, MEL6, MEL7, MEL8, MEL9 and MEL10 or the flanking region thereof.

The primers or probes may comprise a sequence selected from the group comprising SEQ ID NOs: 5, 6, 15, 16, 25, 26, 27, 28, 29, 30, 31, 32, 33, 48 and 49 or combinations thereof, or a sequence with at least 80%, 85%, 90%, 95%, 98% or more sequence identity to a sequence with the sequence of SEQ ID NOs: 5, 6, 15, 16, 25, 26, 27, 28, 29, 30, 31, 32, 33, 48 and 49.

The method of this aspect of the invention may be used with one or more, of the primer pairs of SEQ ID NO: 5 and SEQ ID NO: 6, SEQ ID NO: 15 and SEQ ID NO: 16, SEQ ID NO: 31 and SEQ ID NO: 26, SEQ ID NO: 32 and SEQ ID NO: 28, SEQ ID NO: 29 and SEQ ID NO: 30, SEQ ID NO: 25 and SEQ ID NO: 28, SEQ ID NO: 34 and SEQ ID NO: 6, SEQ ID NO: 48 and SEQ ID NO: 49, or sequences with at least 80%, 85%, 90%, 95%, 98% or more sequence identity to the sequence of the SEQ ID NOs listed and wherein the sequences produce the same PCR product as primers of the specified SEQ ID NOs.

A stable yeast strain may be determined by the molecular weight and/or length and/or quantity and/or sequence of the amplification product of the method of the invention substantially matching a standard. The standard may be DNA from the expected strain.

An unstable yeast strain may be determined by the amplification product not substantially matching the molecular weight and/or length and/or quantity and/or sequence of the standard. An unstable yeast strain may be determined by the failure to produce an amplification product.

According to another aspect of the invention, there is provided a composition comprising one or more oligonucleotides having a sequence selected from the group comprising SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35. 36. 38, 39, 41, 42, 43, 46, 47, 48, and 49, or a sequence with at least 80%, 85%, 90%, 95%, 98% or more sequence identity to the sequence of the SEQ ID NOs listed.

According to a further aspect, the invention provides a kit for determining the strain or strains of yeast in a sample comprising two or more primers or probes directed to two or more target sequences in the yeast nucleic acid, wherein at least one or the primers or probes is directed to a target sequence in a gene, or the flanking region of a gene, in the yeast mitochondrial DNA.

According to a yet further aspect, the invention provides a kit for determining the stability of a strain of yeast in a sample comprising two or more primers or probes directed to two or more target sequences in the yeast nucleic acid, wherein at least one or the primers or probes is directed to a target sequence in a gene, or the flanking region of a gene, in the yeast mitochondrial DNA or at a chromosome subtelomeric region.

A kit of the invention may be suitable for use with PCR.

A kit according to the invention which further comprises a PCR reagent. The PCR reagent may be one or more of a DNA polymerase, a DNA polymerase cofactor, a PCR buffer and one or more deoxyribonucleotide-5′-triphosphates.

Preferably the primers or probes according to any aspect of the invention comprise 12 to 60 nucleotides, more preferably, from 15 to 45 nucleotides.

The primers or probes used in the present invention are selected to be “substantially complementary” to the different strands of each specific sequence to be amplified or detected respectively. This means that they must be sufficiently complementary to hybridize with their respective strands to form the desired hybridized products and wherein the primers may be extendable by a DNA polymerase. In the preferred and most practical situation, the primer or probe has exact complementarity to the target nucleic acid.

The kit may also include instructions to use kit, for example, the instruction may include the PCR conditions to use and/or the amount of sample nucleic acid, and/or primers, and/or other reagents to use.

The kit may also include details of the size of amplification product to expect if a sample contains a particular strain of yeast, or if a strain of yeast is stable or unstable. This may be given by way of a chart or image of a representative electrophoresis gel.

The kit may also include control primers or control probes.

The PCR primers and/or PCR reagents may be provided in a mixture or separately. Two or more probes may be provided in a mixture or separately.

The kit may include a control nucleic acid sample and primers to be used to ensure the PCR conditions are correct. The kit may include a control nucleic acid sample and probes to be used to ensure the probe hybridisation and/or detection conditions are correct.

The kit may comprise further reagents for detecting any PCR amplification product.

The kit may also include instructions regarding how to extract nucleic acid from a sample for analysis. The kit may also include reagents to facilitate nucleic acid extraction.

A kit to determine the strain or strains of yeast in a sample may comprise primers or probes directed to one or more of the following genes, or the flanking sequences thereof, COB, COX1, COX2, COX3, ATP8, ATP6, ATP9, VAR1 and RPM1 or combinations thereof. The kit may also or alternatively include one or more primers or probes directed to all or at least part of one or more of the yeast genes selected from the group comprising the TIR genes, the DAN genes, the FLO genes, the ECM genes, the BUD genes, the KEL genes, the MNT genes, the SED genes, the MEL genes, SUC genes, the ATF genes, the GST genes, the GAL genes, MAL1-4, CYC1, CYC2, CYC3, CBP1, CBP2, CBP3, CBP4, and CBT1 or combinations thereof.

The kit may include one or more primers or probes selected from the group comprising SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 38, 39, 41, 42, and 43, or combinations thereof, or a sequence with at least 80%, 85%, 90%, 95%, 98% or more sequence identity to a sequence with the sequence of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 38, 39, 41, 42, and 43. The kit may comprise one or more of the primer pairs of the primer pairs of SEQ ID NO: 1 and SEQ ID NO: 2, SEQ ID NO: 1 and SEQ ID NO: 3, SEQ ID NO: 1 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 15 and SEQ ID NO: 16, SEQ ID NO: 17 and SEQ ID NO: 18, SEQ ID NO: 19 and SEQ ID NO: 20, SEQ ID NO: 21 and SEQ ID NO: 22, SEQ ID NO: 23 and SEQ ID NO: 24, SEQ ID NO: 31 and SEQ ID NO: 26, SEQ ID NO: 32 and SEQ ID NO: 28, SEQ ID NO: 29 and SEQ ID NO: 30, SEQ ID NO: 25 and SEQ ID NO: 28, SEQ ID NO: 28 and SEQ ID NO: 29 and SEQ ID NO: 27 and SEQ ID NO: 33, SEQ ID NO: 35 and SEQ ID NO: 36. SEQ ID NO: 38 and SEQ ID NO: 39, SEQ ID NO: 41 and SEQ ID NO: 42, and SEQ ID NO: 41 and SEQ ID NO: 43, or sequences with at least 80%, 85%, 90%, 95%, 98% or more sequence identity to the sequence of the SEQ ID NOs listed and produce the same PCR product as primers of the specified SEQ ID NOs. Preferably two or more of the following primer pairs are used in the kit SEQ ID NO: 1 and SEQ ID NO: 3, SEQ ID NO: 1 and SEQ ID NO: 4, SEQ ID NO: 5 and SEQ ID NO: 6, SEQ ID NO: 17 and SEQ ID NO: 18, SEQ ID NO: 19 and SEQ ID NO: 20, SEQ ID NO: 21 and SEQ ID NO: 22, SEQ ID NO: 23 and SEQ ID NO: 24, SEQ ID NO: 31 and SEQ ID NO: 26, SEQ ID NO: 32 and SEQ ID NO: 28, SEQ ID NO: 29 and SEQ ID NO: 30, and SEQ ID NO: 25 and SEQ ID NO: 28, SEQ ID NO: 35 and SEQ ID NO: 36. SEQ ID NO: 38 and SEQ ID NO: 39, SEQ ID NO: 41 and SEQ ID NO: 42, and SEQ ID NO: 41 and SEQ ID NO: 43 or sequences with at least 80%, 85%, 90%, 95%, 98% or more sequence identity to the sequence of the SEQ ID NOs listed, and wherein the sequences produce the same PCR product as primers of the specified SEQ ID NOs.

In a kit to determine the stability of a strain of yeast in a sample primers or probes directed to one or more of the following genes, or flanking sequences thereof, COB, COX1, COX2, COX3, ATP8, ATP6, ATP9, VAR1, RPM1, ECM34, SUC1, SUC3, SUC4, SUC5, SUC7, MAL1, MAL2, MAL3, MAL4, MEL2, MEL3, MEL4, MEL5, MEL6, MEL7, MEL8, MEL9 and MEL10 may be provided

The kit may comprise one or more primers or probes having a sequence selected from the group comprising SEQ ID NOs: 5, 6, 15, 16, 25, 26, 27, 28, 29, 30, 31, 32, 33, 34, 46, 47, 48, and 49 or combinations thereof, or a sequence with at least 80%, 85%, 90%, 95%, 98% or more sequence identity to a sequence with the sequence of SEQ ID NOs: 5, 6, 15, 16, 25, 26, 27, 28, 29, 30, 31, 32, 33, 34, 46, 47, 48, and 49.

The kit may comprise one or more of the primer pairs of SEQ ID NO: 5 and SEQ ID NO: 6, SEQ ID NO: 15 and SEQ ID NO: 16, SEQ ID NO: 31 and SEQ ID NO: 26, SEQ ID NO: 32 and SEQ ID NO: 28, SEQ ID NO: 29 and SEQ ID NO: 30, SEQ ID NO: 25 and SEQ ID NO: 28, SEQ ID NO: 34 and SEQ ID NO: 6, SEQ ID NO: 46 and 47, and SEQ ID NO: 48 and 49, or sequences with at least 80%, 85%, 90%, 95%, 98% or more sequence identity to the sequence of the SEQ ID NOs listed and wherein the sequences produce the same PCR product as primers of the specified SEQ ID NOs.

According to a further aspect, the invention provides a method of fermentation, for example of beer, comprising performing the method of the invention at any stage during the fermentation process. The method of the invention may be performed on a sample of the yeast obtained before propagation, a sample obtained during or after propagation, a sample obtained at any point during the fermentation, or a sample of the end product.

According to another aspect, the invention provides a method of fermentation, for example in the production of a beer, comprising taking a sample of the fermentation mixture during fermentation, performing the method of the invention to either determine what yeast is present or to determine the genetic stability of the yeast, deciding of the basis of the results whether to proceed with the fermentation, and/or whether to change the yeast, and/or whether to redirect the product for a different use.

According to another aspect, the invention provides a method of analysing a yeast containing sample comprising using a probe or primer in said analysis;
wherein the probe or primer is capable of hybridising to mitochondrial DNA of a given yeast, if said yeast is present in said sample.

The probe or primer may not be capable of hybridising to mitochondrial DNA of a further yeast, if present, in said sample.

The given yeast may be S. pastorianus. The further yeast may be S. cerevisiae. Alternatively, the given yeast may be S. cerevisiae. The further yeast may be S. pastorianus.

The method according to any aspect of the invention may be used to determine whether or not a yeast containing sample contains an undesired yeast. The method may be for use in checking the quality of a sample intended for use in subsequent fermentation involving yeast. The method may comprise the step of performing fermentation using said yeast if the quality is acceptable or aborting fermentation if the quality is not acceptable.

According to another aspect, the invention provides a probe or primer suitable for use in a method of the invention.

The probe or primer may preferentially hybridise to mtDNA of S. pastorianus, or preferentially hybridise to mtDNA of S. cerevisiae.

The probe or primer may be at least 8 nucleotides in length. The probe or primer may be less than 30 nucleotides in length.

The probe or primer may preferentially hybridise to any of the genes selected from COB, COX1, COX2, COX3, ATP8, ATP6, ATP9, VAR1 and RPM1. The probe or primer may preferentially hybridise to any of the sequences selected from the group comprising SEQ ID NO: 37, 40, 44, 45, 50, 51, 54, 55, 58, 59, 60, 61, 62, 63, 64, 65, 66, 67, 68, 69, 70, 71, and 72, or complements thereof.

The probe or primer may preferentially hybridise to SEQ ID NO: 50 or SEQ ID NO: 54, or complements thereof, and optionally does not hybridise to SEQ ID NO: 51 or SEQ ID NO: 55, or complements thereof. Alternatively, the probe or primer may preferentially hybridise to SEQ ID NO: 51 or SEQ ID NO: 55, or complements thereof, and optionally does not hybridise to SEQ ID NO: 50 or SEQ ID NO: 54, or complements thereof.

The probe or primer may preferentially hybridise to any of SEQ ID NOS: 58, 59 or 60, or complements thereof, and optionally does not hybridise to any of SEQ ID NOS: 61, 62, 63, 64, 65, or 66, or complements thereof. Alternatively, the probe or primer may preferentially hybridise to any of SEQ ID NOS: 61, 62, 63, 64, 65, or 66, or complements thereof, and optionally does not hybridise to any of SEQ ID NOS: 58, 59 or 60, or complements thereof.

The probe or primer may preferentially hybridise to any of SEQ ID NOS: 67 or 68, or complements thereof, and optionally does not hybridise to any of SEQ ID NOS: 69, 70, 71, or 72, or complements thereof. Alternatively, the probe or primer may preferentially hybridise to any of SEQ ID NOS: 69, 70, 71, or 72, or complements thereof, and optionally does not hybridise to any of SEQ ID NOS: 67 or 68, or complements thereof.

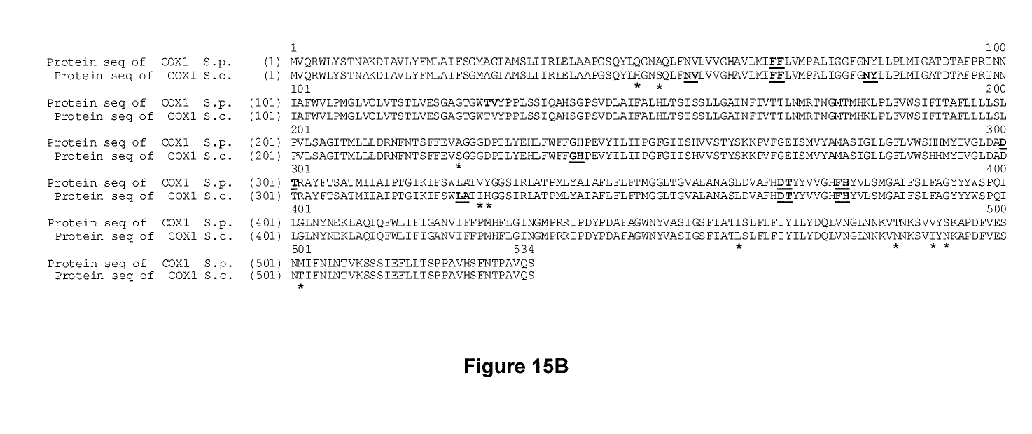

The probe or primer may preferentially hybridise to part of any of SEQ ID NOS: 50, 51, 54, 55, 58, 59, 60, 61, 62, 63, 64, 65, 66, 67, 68, 69, 70, 71, or 72, or complements thereof, at one or more nucleotide locations indicated by an asterisk (*) in any of FIG. 15A, 16A, 17 or 18. For example the probe or primer may preferentially hybridise to a nucleotide location marked by an asterisk in FIG. 15A, which comprises nucleotide number 147 of SEQ ID NO: 50.

Reference made to “hybridising” may refer to hybridising under stringent conditions. The term “preferential hybridisation”, or similar, is intended to refer to the greater likelihood of hybridisation of a primer or probe to a particular sequence or target region relative to another sequence or target region. Preferential hybridisation may be determined under stringent conditions. Stringency conditions for hybridization refers to conditions of temperature and buffer composition which permit hybridisation of the primers or probes, or of complementary sequence to each other, for example in PCR, wherein conditions determine whether sequences of certain identity are capable of hybridisation to each other. Sequences less similar to each other will hybridise under moderate stringency conditions. High stringency requires the hybridising sequences to be identical, or almost identical, for hybridisation to occur.

The term “hybridising” used herein may refer to hybridising under moderate to high stringency conditions readily determined by the skilled person.

The method of determining the genetic stability of a yeast strain in a sample, may comprising screening the yeast by digestion of the yeast nucleic acid with a restriction enzyme, which specifically cuts the nucleic acid between a guanine nucleotide and a cytosine nucleotide (ĜC) to provide an RFLP pattern (Restriction Fragment Length Polymorphism),
wherein the RFLP pattern of the yeast nucleic acid is compared to a known conserved RFLP pattern from a yeast that is not unstable, and wherein the observation of a significant difference in RFLP pattern indicates an unstable yeast strain. An unstable yeast strain may otherwise be known as a “petite mutant”.

The yeast nucleic acid may comprise whole cell DNA (including mtDNA), or mtDNA.

A conserved RFLP pattern of a stable yeast strain may, for example, substantially match the fragment sizes listed in Table 4 when cut with restriction enzymes HaeIII or Hinfl.

The method of any aspect of the invention herein may be for use in brewing.

“Sequence identity” used herein may refer to the comparison of two sequences using, for example using sequence analysis software BLASTN or BLASTP (available at www.ncbi.nlm.nih.gov/BLAST/). BLASTN or BLASTP default parameters may be used for determining sequence identity.

It will be appreciated that optional features applicable to one aspect or embodiment of the invention can be used in any combination, and in any number. Moreover, they can also be used with any of the other aspects or embodiments of the invention in any combination and in any number where appropriate.

Embodiments of the invention will now be described, by way of example only, with reference to the accompanying drawings, in which:—

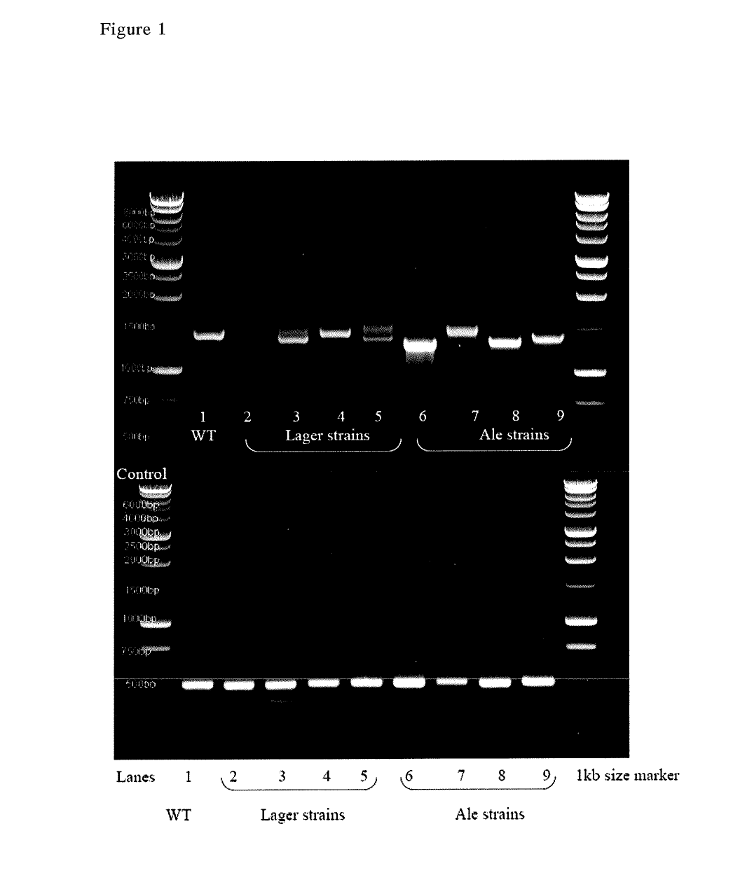

FIG. 1—shows an electrophoresis profile of PCR amplification of the TIR4 gene. Lane 1—WT (S288c); Lane 2—UNYC3; Lane 3—UNYC2; Lane 4—NCYC1116; Lane 4 UNYC1; Lane 5—UNYC7; Lane 6—UNYC8; Lane 7—NCYC1119; Lane 8—KS1 and Lane 9—NCYC2593;

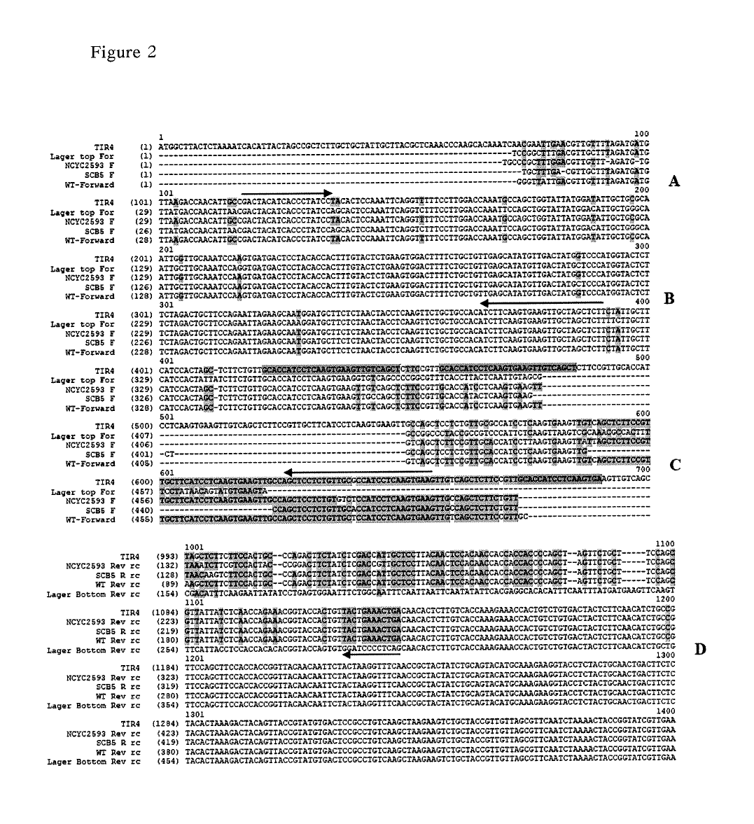

FIG. 2—shows sequence alignments of the bands isolated in FIG. 1. Primer A+C and A+D combinations will differentiate ale and lager strains. Primer combination A+B is used as a control;

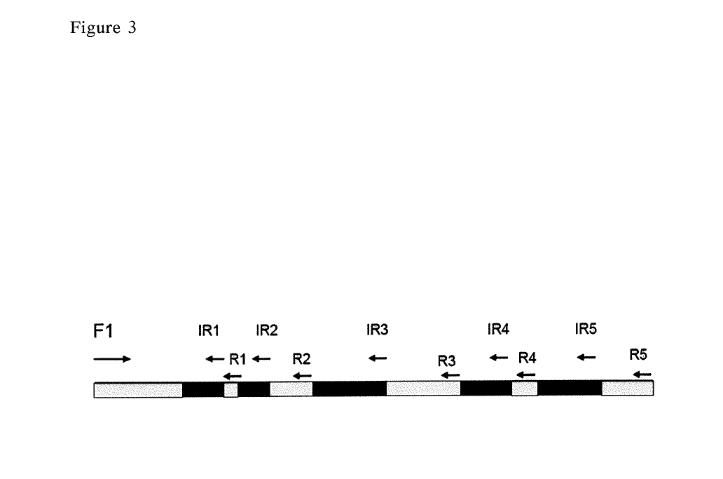

FIG. 3—is a schematic diagram representing the COB gene in S. cerevisiae. Introns are represented as dark regions (F=forward primer, R=reverse primer);

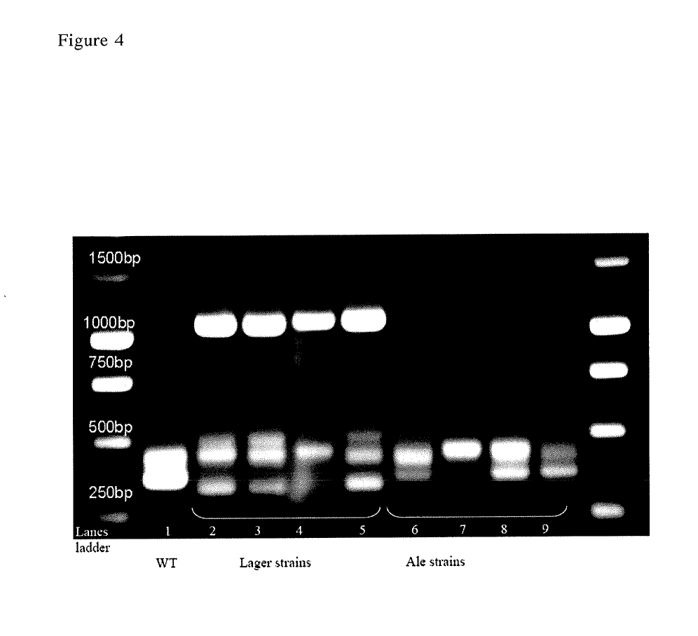

FIG. 4—shows an electrophoresis profile of the amplified COB gene using primers F1 and R1. Lane 1—WT (S288c); Lane 2—UNYC3; Lane 3—UNYC2; Lane 4—NCYC1116; Lane 4 UNYC1; Lane 5—UNYC7; Lane 6—UNYC7; Lane 7—NCYC1119; Lane 8—KS1 and Lane 9—NCYC2593;

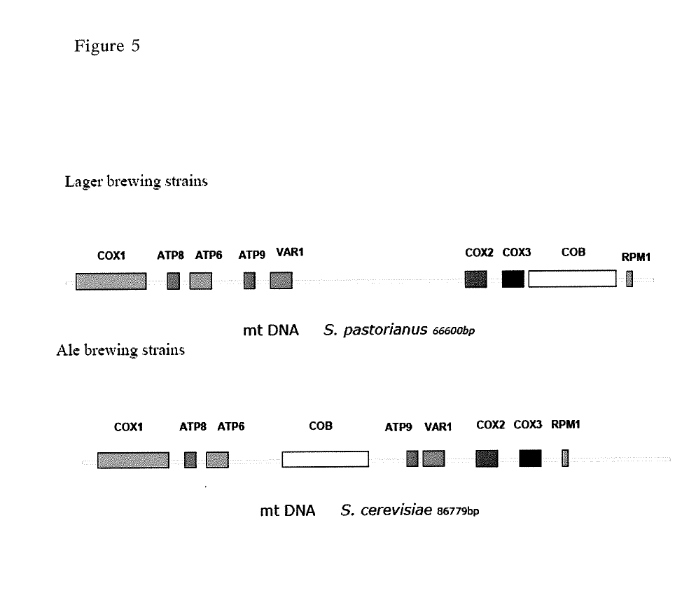

FIG. 5—is a schematic representation of the mtDNA sequences of S. pastorianus and S. cerevisiae yeast strains;

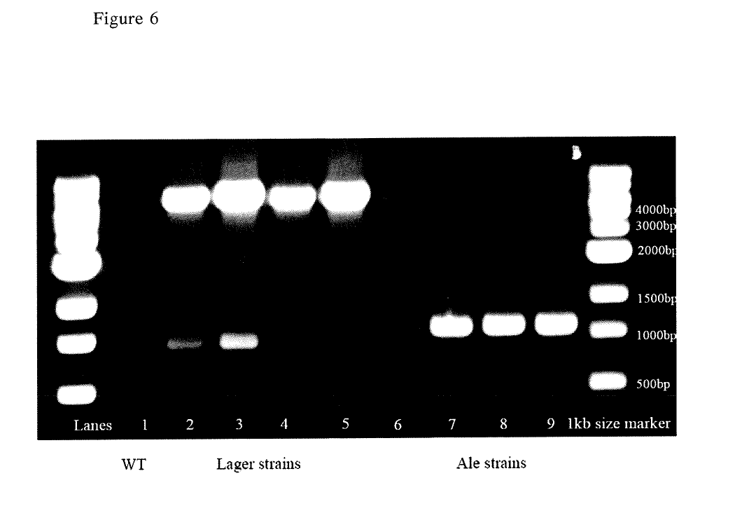

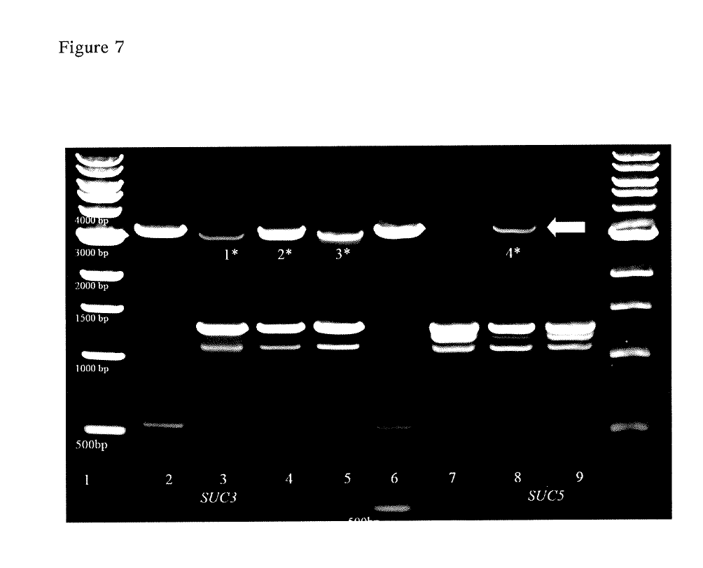

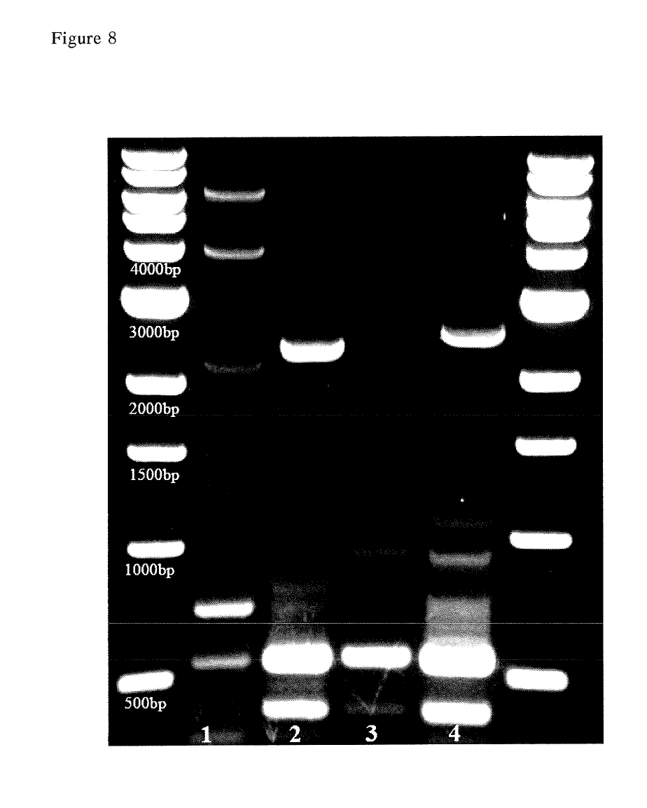

FIG. 6—shows an electrophoresis profile of MEL1 gene. Lane 1—WT (S288c); Lane 2—UNYC2; Lane 3—UNYC1; Lane 4—UNYC3; Lane 5—UNYC4; Lane 6—UNYC7; Lane 7—UNYC8; Lane 8—UNYC9 and Lane 9—UNYC10;

FIG. 7—shows an electrophoresis profile of SUC3 and SUC5 genes. Lane 1—1 kb ladder: Lane 2—WT (S288c); Lane 3—UNYC1; Lane 4—UNYC2; Lane 5—NCYC1116; Lane 6—WT (S288c); Lane 7—UNYC1; Lane 8—UNYC2; Lane 9—NCYC1116;

FIG. 8—shows an electrophoresis profile of FLO1 gene. right and left hand lanes—1 kb ladder: Lane 1—WT (S288c); Lane 2—UNYC1; Lane 3—UNYC2; Lane 4—NCYC1116;

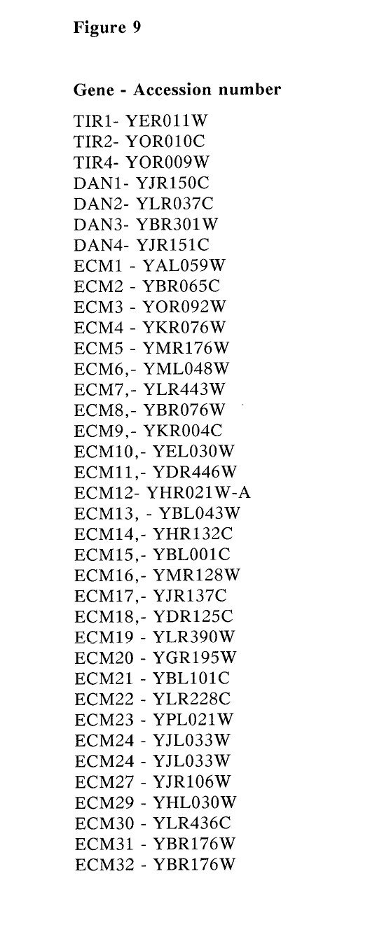

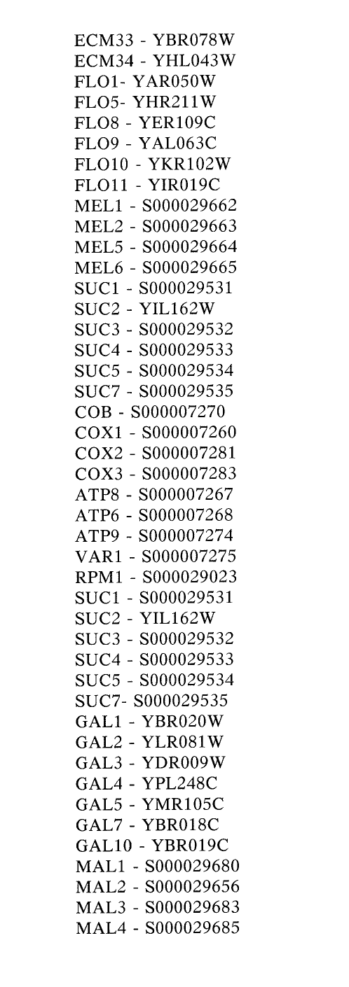

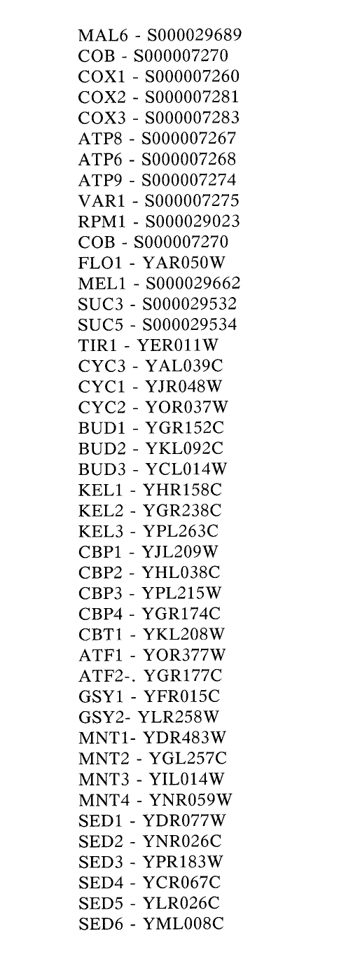

FIG. 9—shows the GenBank accession number for all the genes referred to herein:

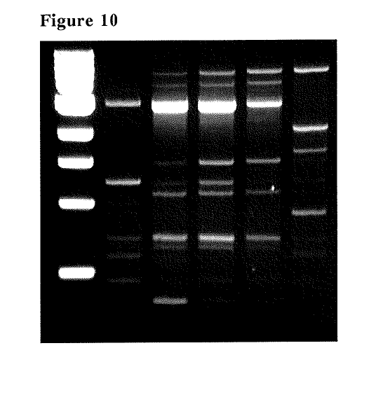

FIG. 10—shows an electrophoresis profile of the COB gene to identify the genomic stability of petite mutants of strain UNYC2. From right to left—Lane 1—1 kb ladder; Lane 2—WT (S288c); Lane 3—UNYC1 parent strain; Lane 4—UNYC1 petite mutant strain; Lane 5—UNYC2 parent strain; Lane 6—UNYC2 petite mutant strain;

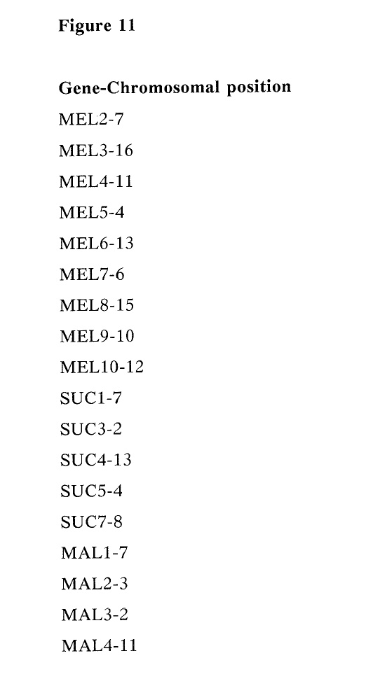

FIG. 11—details a number of subtelomerically located genes and their chromosomal position;

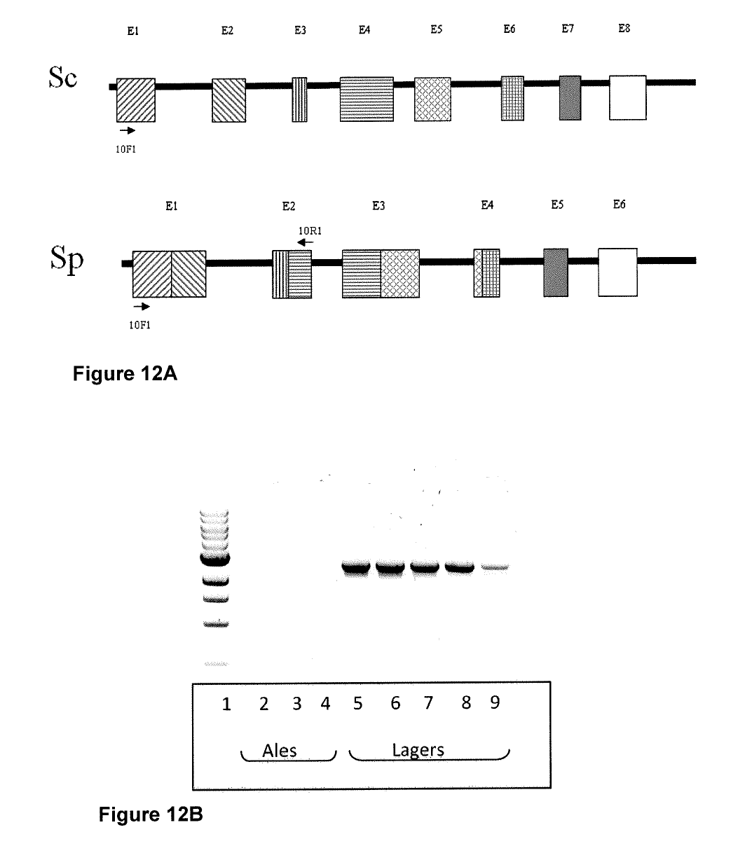

FIG. 12A—shows a schematic representation of the position of primers used in standard PCR to differentiate ale and lager strains using primer 10F1 and 10R1. (Note that in S. cerevisiae (Sc) 10R1 primer was not present); FIG. 12B-Electrophoresis profile of ale and lager yeast strain differentiation by standard PCR using COX1 gene. Lane 1: 1 kb size marker; Lane 2; S288C (WT); Lane 3: NCYC1119; Lane 4: NCYC 2593; Lane 5: W34 UON; Lane 6: LBY1; Lane 7: LBY2; Lane 8: NCYC1116 and Lane 9: LBY11). Primer pair 10F1 and 10R1 was designed for mitochondrial genomic sequence of W34UON sequence;

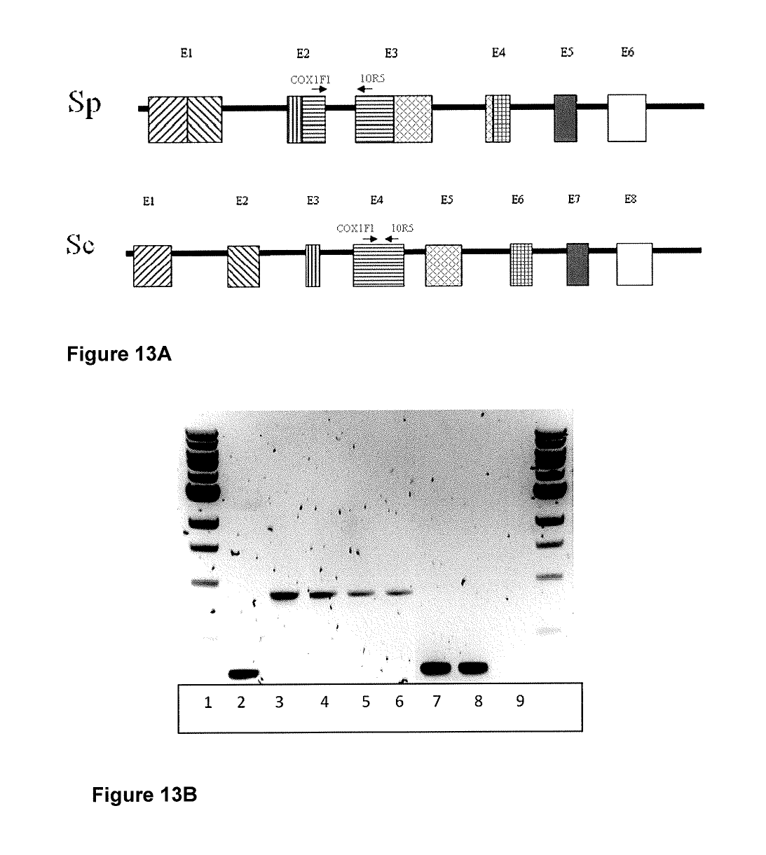

FIG. 13A—shows a schematic representation of the position of primers used in standard PCR to differentiate ale and lager strains using primer COX1F1 and 10R5; 13B—Electrophoresis profile of ale and lager yeast strain differentiation by standard PCR using COX1 gene. Lane 1 and 10: 1 kb size marker; Lane 2; S288C (WT); Lane 3: W34 UON; Lane 4: LBY11; Lane 5:LBY1; Lane 6: LBY2; Lane 7:NCYC2593; Lane 8: ABY6 and Lane 9: Negative Control). Lanes 2, 7 and 8 correspond to the ale type strains and lanes 3-6 correspond to lager-type strains. Primer pair COX1F1 and 10R5 was designed for mitochondrial genomic sequence of W34UON sequence;

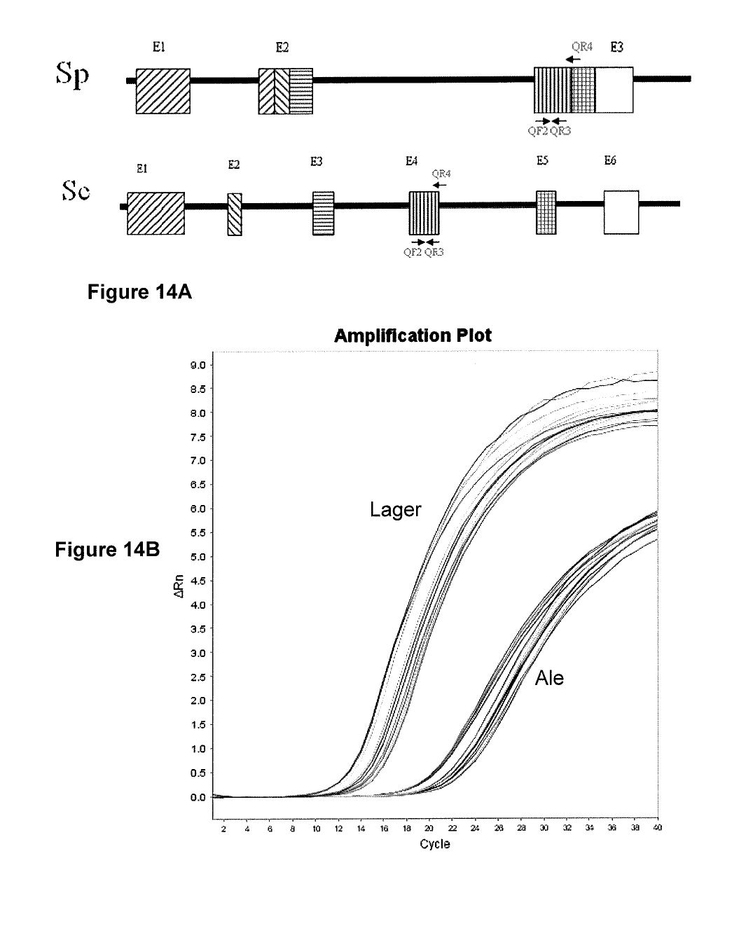

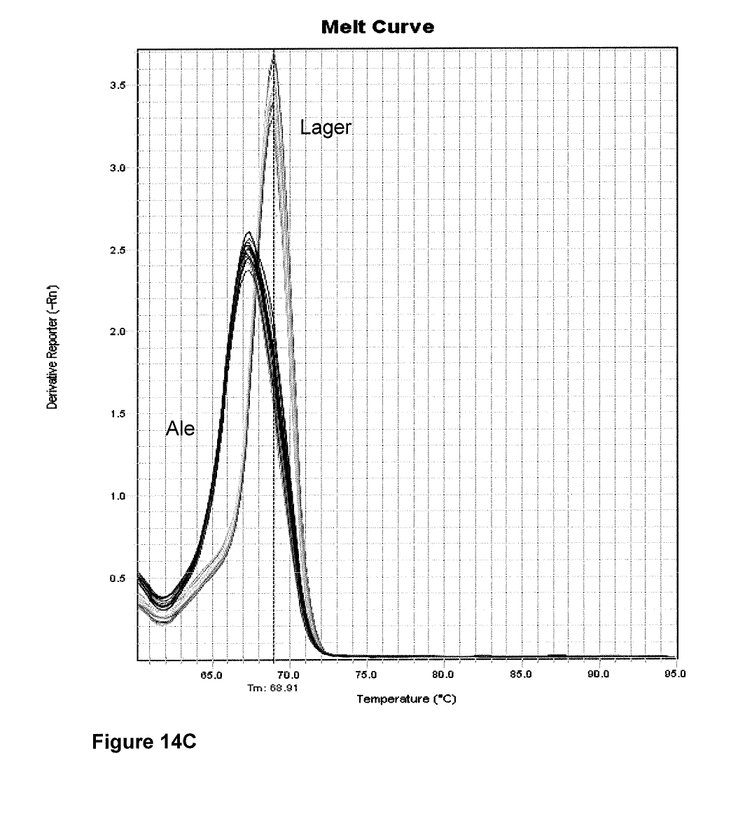

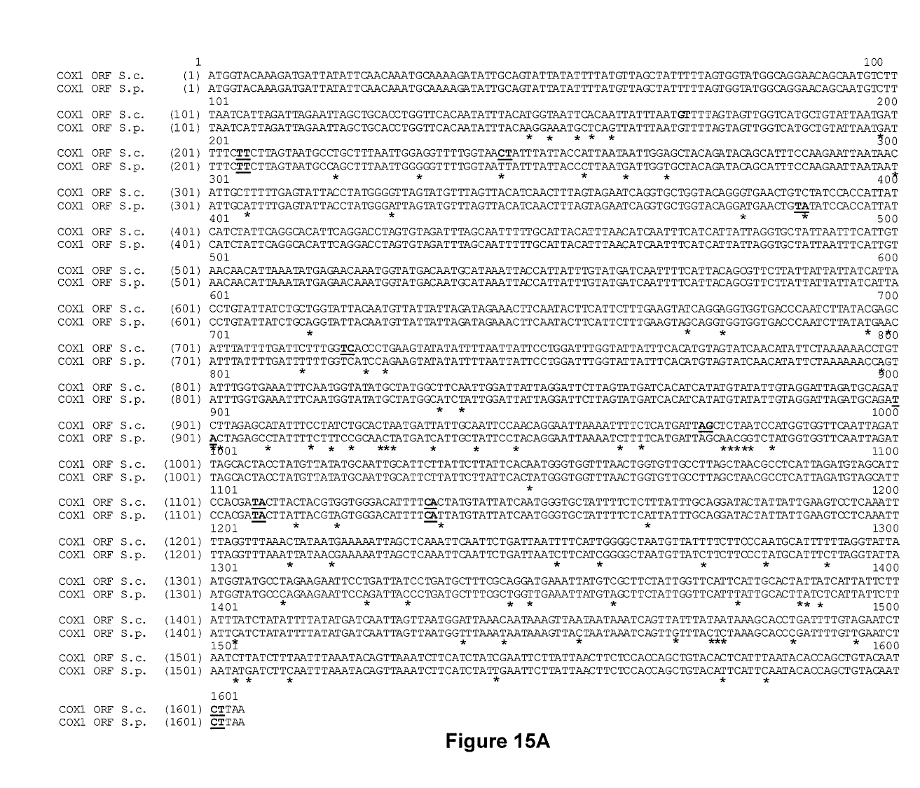

FIG. 14A—shows a schematic representation of the position of primers used in Real-Time PCR to differentiate ale and lager strains using COB gene; FIG. 14B—shows an amplification plot of the COB gene; and FIG. 14C—shows a melting curve of the Real-Time PCR product of FIG. 14B;

FIG. 15A—shows a sequence alignment of S. cerevisiae (Sc) (SEQ ID NO: 50) and S. pastorianus (Sp) (SEQ ID NO: 51) COX/open reading frame. Exon boundaries are underlined in black. Sequence differences are highlighted by an asterisk; FIG. 15B—shows a sequence alignment of S. cerevisiae (Sc) (SEQ ID NO: 52) and S. pastorianus (Sp) (SEQ ID NO: 53) COX1 open reading frame translation. Exon boundaries are underlined in black. Sequence differences are highlighted by an asterisk;

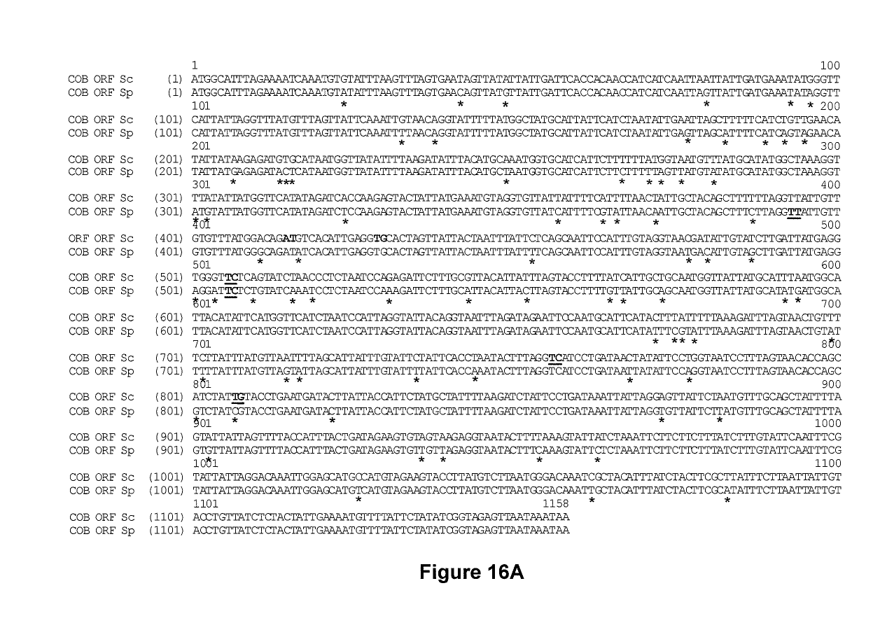

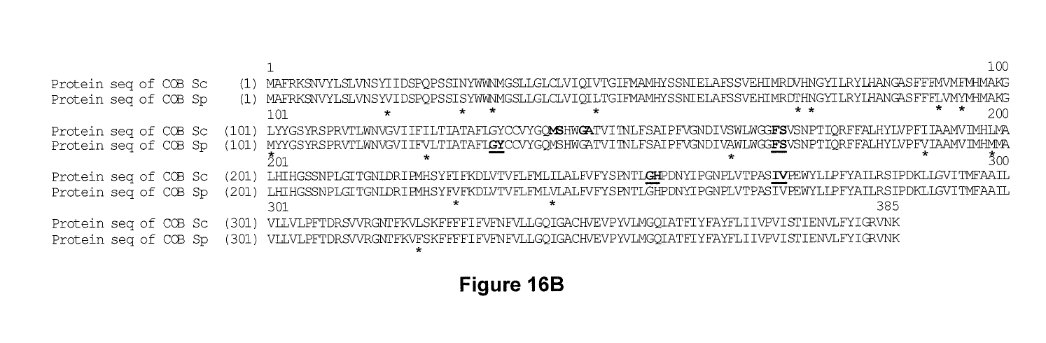

FIG. 16A—shows a sequence alignment of S. cerevisiae (Sc) (SEQ ID NO: 54) and S. pastorianus (Sp) (SEQ ID NO: 55) COB open reading frame. Exon boundaries are underlined in black. Sequence differences are highlighted by an asterisk; FIG. 16B—shows a sequence alignment of S. cerevisiae (Sc) (SEQ ID NO: 56) and S. pastorianus (Sp) (SEQ ID NO: 57) COB open reading frame translation. Exon boundaries are underlined in black. Sequence differences are highlighted by an asterisk;

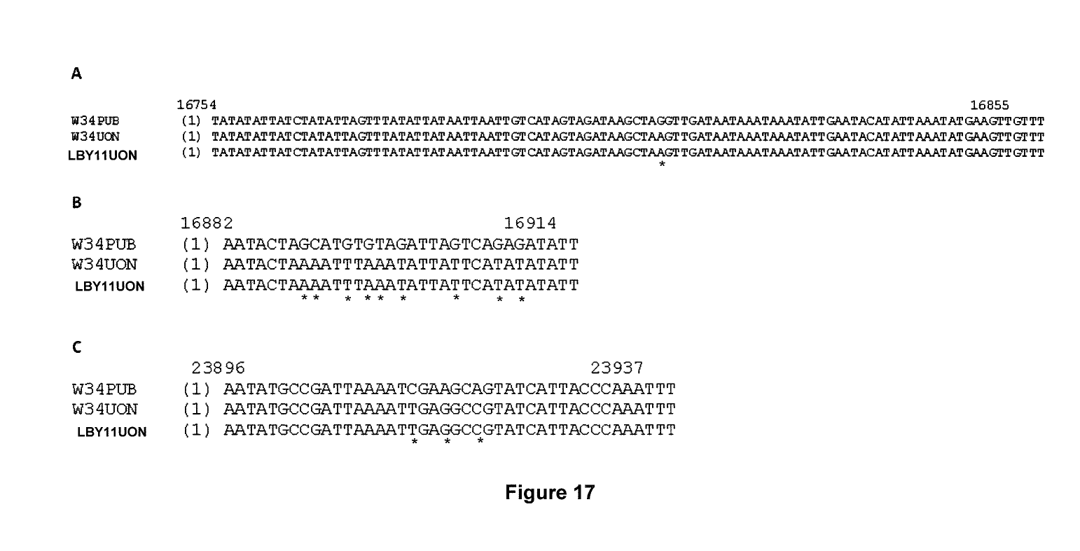

FIG. 17—shows a sequence alignment of regions A, B and C of W34PUB, W34UON and LBY11UON, to identify the sequence differences to develop primers for strain differentiation. W34PUB “region A”=SEQ ID NO: 58, W34PUB “region B”=SEQ ID NO: 59, W34PUB “region C”=SEQ ID NO: 60; W34UON “region A”=SEQ ID NO: 61, W34UON “region B”=SEQ ID NO: 62, W34UON “region C”=SEQ ID NO: 63; LBY11UON “region A”=SEQ ID NO: 64, LBY11UON “region B”=SEQ ID NO: 65, LBY11UON “region C”=SEQ ID NO: 66. Sequence differences are highlighted by an asterisk. Numbers refer to the numbers of W34PUB sequence Accession No: EU852811. The whole mitochondrial genome sequence of W34PUB and W34UON is 70578 bps;

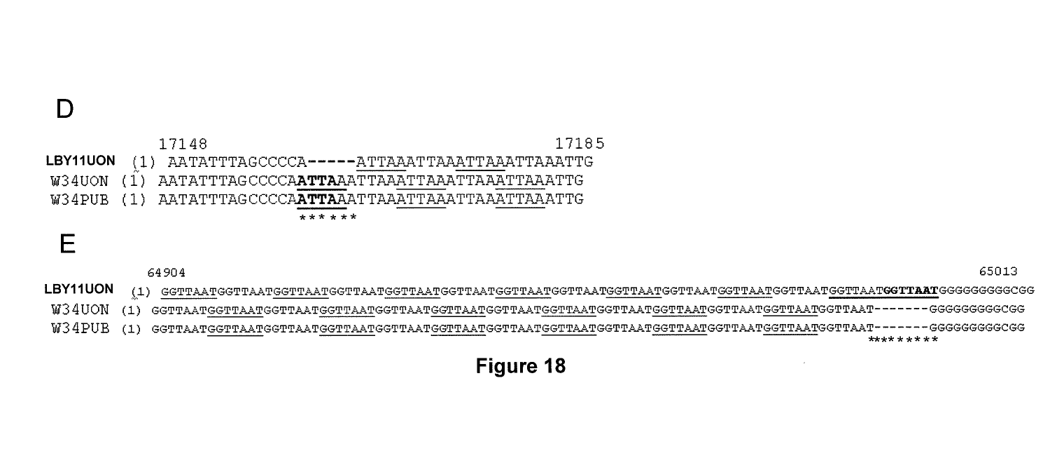

FIG. 18—shows a sequence alignment of regions D and E of the W34PUB, W34UON and LBY11UON sequences, to identify differences. LBY11UON “region D”=SEQ ID NO: 67, LBY11UON “region E”=SEQ ID NO: 68; W34UON “region D”=SEQ ID NO: 69, W34UON “region E”=SEQ ID NO: 70; W34PUB “region D”=SEQ ID NO: 71, W34PUB “region E”=SEQ ID NO: 72. Numbers refer to LBY11UON mitochondrial genome sequence. The whole mitochondrial genome sequence of LBY11UON is 70579 bps;

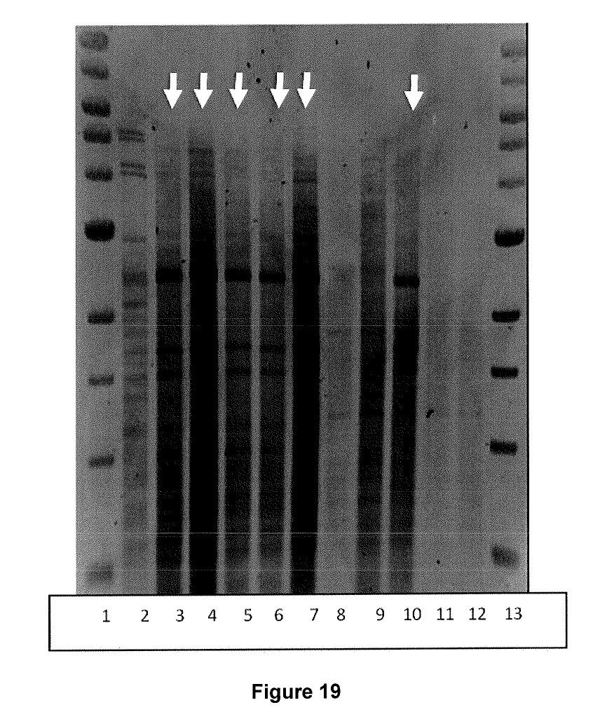

FIG. 19—shows a mitochondrial DNA restriction profile after digestion of total DNA using HaeIII restriction enzyme. Lane 1 and 13: 1 kb DNA marker; Lane 2 LBY11UON; Lane 3 to 12 LBY11UON petite isolated from brewery fermentations. Arrows indicate the predominant petite RFLP pattern;

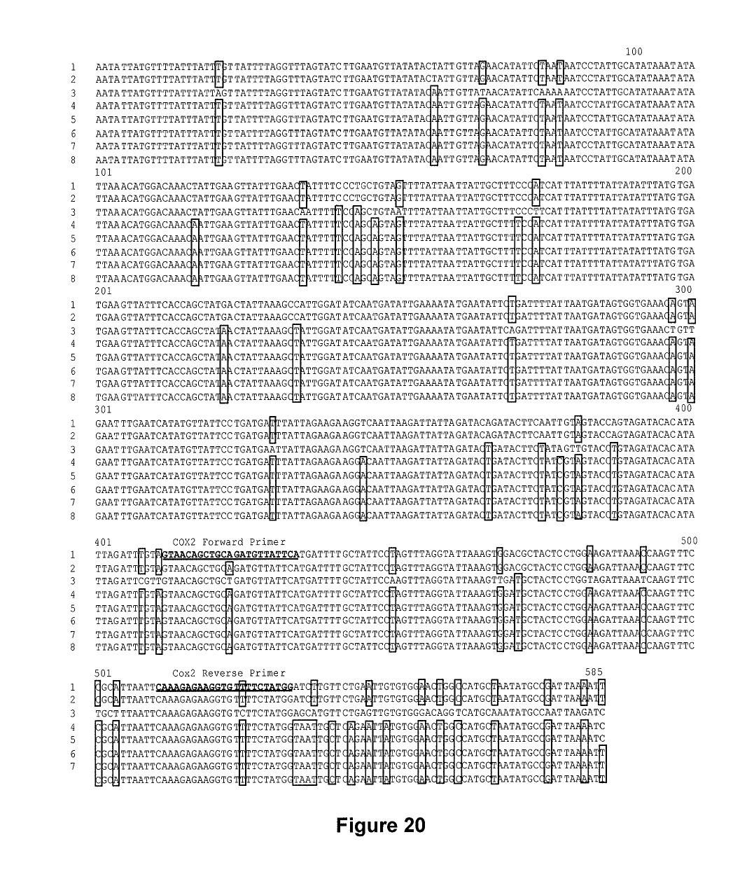

FIG. 20—shows an alignment of partial COX2 sequences from 8 strains of yeast. 1; S. bayanus AF442211 (SEQ ID NO: 73). 2; S. uvarum AY130328 (SEQ ID NO: 74). 3; S. cerevisiae ef639728 (SEQ ID NO: 75). 4: S. bayanus ef639726 (SEQ ID NO: 76). 5; S. pastorianus-ef639727 (SEQ ID NO: 77). 6; S. carlsbergensis AY130326 (SEQ ID NO: 78). 7; S. pastorianus AF442212 (SEQ ID NO: 79). 8; S. monacensis AY130325 (SEQ ID NO: 80). Base pair substitutions are highlighted by a box. COX2 forward and reverse primers are underlined;

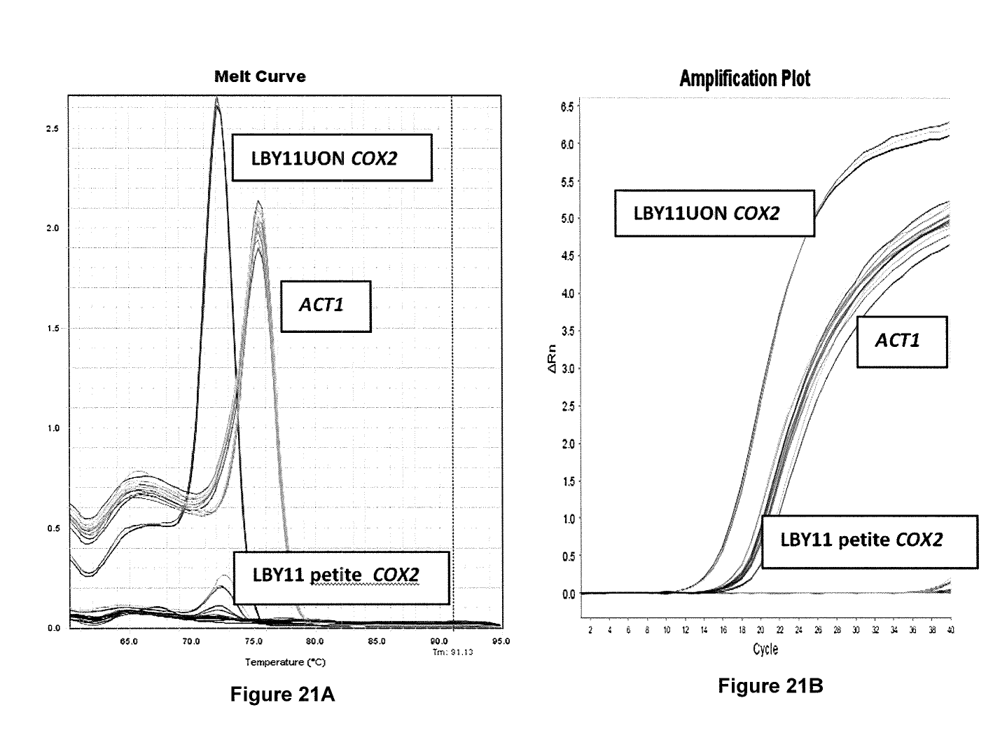

FIG. 21A—shows a melt curve; and FIG. 21B—shows an amplification plot for real time PCR product using COX2 and ACT1 primers (Table 5);

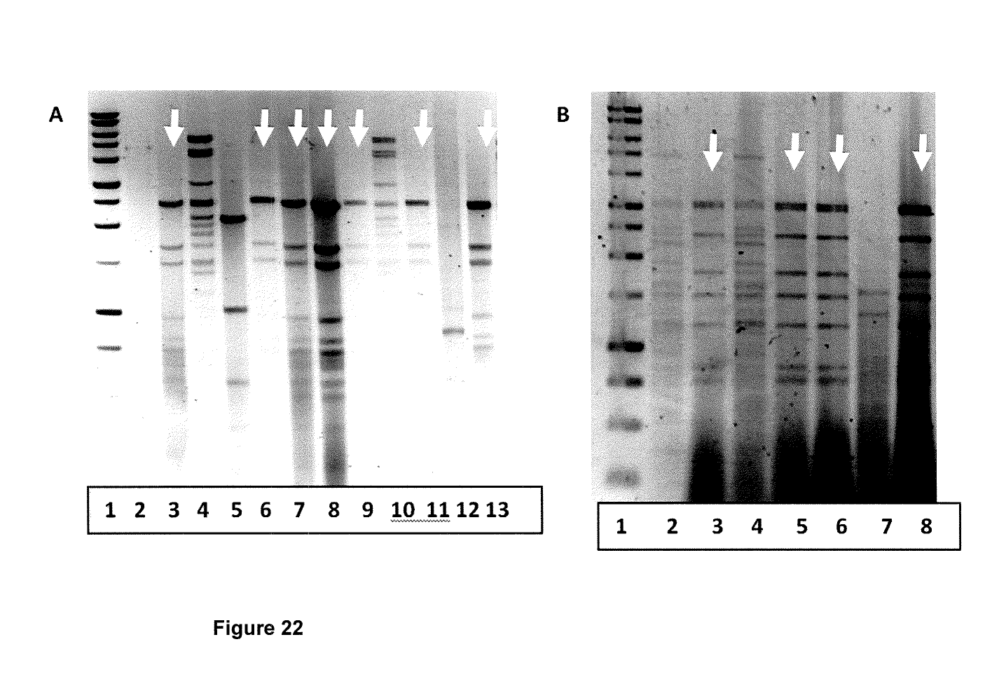

FIGS. 22A and 22B—show a mitochondrial DNA restriction profile after digestion of total DNA using HaeIII (FIG. 22A) HinfI (FIG. 22B) restriction enzyme. FIG. 22A-Lane 1: 1 kb DNA marker; Lane 2, 3, 56, 7, 8, 9, 11, 12, and 13 are brewery petites. Lane 4 and 10: LBY11UON; FIG. 22B-Lane 1: 1 kb DNA marker; Lane 2: LBY11UON; Lane 3 to 8 brewery isolates of petites. Arrows indicate the predominant petite pattern;

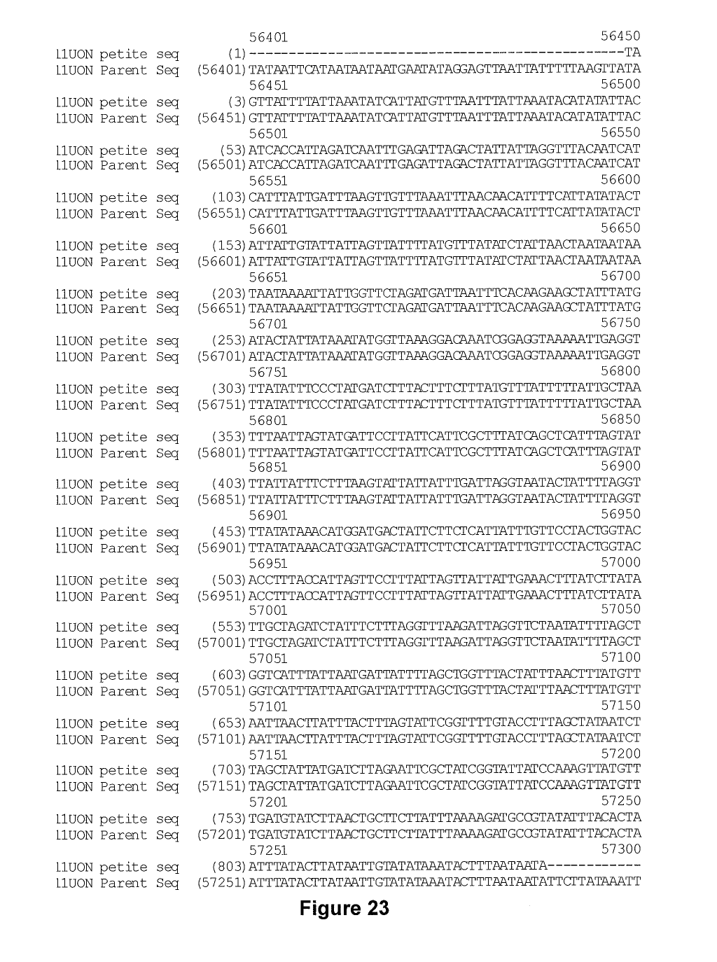

FIG. 23—shows a sequence alignment of the LBY11UON (SEQ ID NO: 81) and LBY11UON partial petite sequence (SEQ ID NO: 82) to identify differences. Numbers refer to LBY11UON mitochondrial genome sequence.

### EXAMPLES

The following examples illustrate the development of multiple primer sets that can be used according to the invention to identify brewing yeast strains rapidly and accurately providing unambiguous and reproducible differentiation of proprietary brewing yeast.

The data presented demonstrates that a number of genes, including the MEL1 gene, can be used to differentiate between ale and lager yeast strains.

The data also demonstrates that mtDNA (mitochondrial DNA) can be used to differentiate lager and ale yeast strains. Primers were designed directed to the mitochondrial gene COB, and those which could be used to differentiate between different brewing yeast strains were identified. One set of oligonucleotides was identified to differentiate ale and lager strains accurately. This was designed across a conserved region of COB gene.

Results were confirmed using the brewing yeast strains available in the University of Nottingham brewing yeast culture collection.

### Identifying Suitable Target Sites for the Design of Specific Molecular Markers

Fourteen ale and lager brewing yeast strains were chosen from the University of Nottingham in house culture collection to develop primers to differentiate the brewing yeast strains.

Strains Used in this Study

| | | |
|---|---|---|
| 1. | UNYC1 | S. carlsbergensis (lager) |
| 2. | UNYC2 | S. cerevisiae (lager) |
| 3. | UNYC3 | S. cerevisiae (sy. pastorianus) (lager) |
| 4. | UNYC4 | S. cerevisiae (sy. pastorianus) (lager) |
| 5. | UNYC5 | S. cerevisiae (sy. pastorianus) (lager) |
| 6. | UNYC6 | S. cerevisiae (sy. pastorianus) (lager) |
| 7. | NCYC1116 | S. carlsbergensis (lager) |
| 8. | UNYC7 | S. cerevisiae (ale) |
| 9. | UNYC8 | S. cerevisiae (ale) |
| 10. | UNYC9 | S. cerevisiae (ale) |
| 11. | UNYC10 | S. cerevisiae (ale) |
| 12. | NCYC 1119 | S. cerevisiae (ale) |
| 13. | NCYC 2593 | S. cerevisiae (ale) |
| 14. | KS1 | S. cerevisiae (ale) |
| 15. | Wild-type | S. cerevisiae (S288c) (used as a control) |

### Nuclear Genes as a Target

The sequence of the five nuclear genes shown in Table 1 were analysed to identify polymorphic regions across the genome. Primers were designed, as indicated in Table 1, to these regions.

TABLE 1

| Gene | Function | Primers 5′-3′ |
|---|---|---|
| TIR1 | Encodes putative cell wall | Forward- |
| (YER011W) | mannoprotein of the | ATGGCTTACTCTAAAATCACA |
| | Srp1p/Tip1p family of | (Seq ID No: 7) |
| | serine-alanine-rich proteins | Reverse- |
| | in S. cerevisiae | GTGCTTTAGCTGCTGTTGC |
| | | (Seq ID No: 8) |
| TIR2 | Encodes putative cell wall | Forward- |
| (YOR010C) | mannoprotein of the | ATGGCTTACATCAAGATC |
| | Srp1p/Tip1p family of | (Seq ID No: 9) |
| | serine-alanine-rich proteins | Reverse- |
| | in S. cerevisiae | TTATAATAACATGGCGGCAGC |
| | | (Seq ID No: 10) |
| TIR4 | Encodes putative cell wall | Forward- |
| (YOR009W) | mannoprotein of the | ATGGCTTACTCTAAAATCACATTA |
| | Srp1p/Tip1p family of | (Seq ID No: 11) |
| | serine-alanine-rich proteins | Reverse- |
| | in S. cerevisiae | TCATAGTAGCATGGCGGCAACAGC |
| | | (Seq ID No: 12) |
| DAN4 | Encodes putative cell | Forward- |
| (YJR151C) | wall mannoprotein in | ATGGTTAATATAAGCATCGTAG |
| | S. cerevisiae | (Seq ID No: 13) |
| | | Reverse- |
| | | CTATCGTTGCTGTTGTCGC |
| | | (Seq ID No: 14) |
| ECM34 | Encodes putative protein | Forward- |
| (YHL043W) | possibly involved in cell | ATGGAGGGCCGCA |
| | wall structure in | (Seq ID No: 15) |
| | S. cerevisiae and considered | Reverse- |
| | as recombination hot spot | CACAGAATACTTTTTTTGTTGA |
| | common to lager yeast | (Seq ID No: 16) |
| | strains | |

### TIR 4

FIG. 1 shows an electrophoresis profile of the amplification products observed when the genomic DNA from a number of yeast strains were subjected to PCR with primers (as detailed in Table 1, Seq ID No. 11 and 12) directed to the TIR4 gene. The PCR conditions used are detailed below. The yeast strains analysed were wild-type (S288c), UNYC3, UNYC2, NCYC1116, UNYC1, UNYC7, UNYC8, NCYC1119, KS1, and NCYC2593 strains. Bands were isolated and confirmed further by sequencing. The sequencing data obtained from the TIR4 gene was used to produce a sequence alignment, as shown in FIG. 2, and to design further primers shown in Table 2, which are directed to a conserved sequence area specific to lager and ale strains. Table 2 also details the results of PCR using these primers which demonstrates that primers A and C can be used to positively identify ale strains, A and D can be used to positively identify lager strains, and A and B can be used as a positive control.

### PCR Conditions

98° C.—30 seconds
35 cycles
98° C.—10 seconds
58° C.—30 seconds
72° C.—1 minute

TABLE 2
Sequences of the oligonucleotide primer pairs
designed within the TIR4 gene which can be used
to identify ale and lager brewing yeast strains.

| Primer | | | |
|---|---|---|---|
| Combination | Sequence 5′- to 3′ | Ale | Lager |
| Primer A | CGA CTA CAT CAC CCT | Yes | Yes |
| | ATC C | Control | Control |
| | (SEQ ID NO: 1) | | |
| Primer B | GCA ACT TCA CTT GAA G | | |
| | (SEQ ID NO: 2) | | |
| Primer A | CGA CTA CAT CAC CCT | Yes | No |
| | ATC C | | |
| | (SEQ ID NO: 1) | | |
| Primer C | GCG CAA CAG AGG AGC | | |
| | (SEQ ID NO: 3) | | |
| Primer A | CGA CTA CAT CAC CCT | No | Yes |
| | ATC C | | |
| | (SEQ ID NO: 1) | | |
| Primer D | CTG AGG GGA TCC | | |
| | (SEQ ID NO: 4) | | |

### A Melabiose Utilisation Gene—MEL1

The primer sequences:

| (Seq ID No: 17) | |
|---|---|
| MEL1F | 5′- TGACTAAATCTGGAAAACCACATGG-3′ |
| (Seq ID No: 18) | |
| MEL1R4 | 5′- CAAATATGCCAACATTGTTGACAG-3′ |

were used with the following PCR conditions
98° C.—30 seconds
35 cycles
98° C.—10 seconds
60° C.—30 seconds
72° C.—2 minutes
Final 72° C.—10 minutes
to amplify the MEL1 gene in the nucleic acid isolated from yeast samples. The results of a comparison of ale (lanes 6, 7, 8, and 9) and lager (2, 3, 4, and 5) strains, using primers 17 and 18, are shown in FIG. 6, in which lager strains produced a band corresponding to 5 kb compared to some of the ale strains which produce an ˜1150 bp band. The presence of the 5 kb band can be used to identify lager yeast strains. Strain UNYC7 (an ale strain—lane 6) did not produce any bands in this PCR reaction, thus this simple PCR will allow the UNYC7 strain to be differentiated from other stains in the collection. The lager strains UNYC1 (lane 1) and UNYC2 (lane 2) produce two extra bands corresponding to 1 kb, thereby allowing these two strains to be differentiated from the rest of the lager strain collection. Further secondary PCR primer may be used to distinguish UNYC1 and UNYC2 strains from each other.

### Invertase Utilisation Genes (SUC3 and SUC5)

FIG. 7 demonstrates that the primer pairs SUC3F/SUC3R and SUC5F/SUC5R (see below for sequences) are able to differentiate the UNYC2 lager yeast strain from other lager yeast strains. More specifically, SUC3F and SUC3R primers are able to differentiate UNYC2 strain from UNYC1 strain, and SUC5F/SUC5R are able to differentiate the UNYC2 strain from the rest of lager strains.

In FIG. 7 the bands identified as *1, *2 *3 and *4 were purified and sequence was analysed. These three bands were different in size which can be also be used to differentiate the three lager strains UNYC1, UNYC2 and NCYC1116.

### Primer Sequences

(Seq ID No: 19)
SUC3F-5′- ATC GAT AGG CAC TGC ACA GTG G-3′
(Seq ID No: 20)
SUC3R-5′- ATG ACA CTG TTT GGG GTT TGC C-3′
(Seq ID No: 21)
SUC5F-5′- ATC GAT AGG CAC TGC ACA GTG G-3′
(Seq ID No: 22)
SUC5R-5′- ATG ACA CTG TTT GGG GTT TGC-3′

### PCR Conditions

98° C.—30 seconds
35 cycles
98° C.—10 seconds
63° C.—30 seconds
72° C.—1 minute
Final—72° C.—10 minutes

### Flocculation Genes

FIG. 8 demonstrates that the primers FLO1F1 and FLOR1 (see below) designed to flanking regions of the FLO1 genes can be used to differentiate between lager strains, such as NCYC116.

The primers FLO1F1 and FLO1R1 were used in a PCR with a number of yeast strains. It was observed that the WT (S288c) strain gave rise to a band with a size of 4.6 kb compared to lager yeast strains. Furthermore strain NCYC116 gave rise to a larger band which can be used to easily differentiate from other strains (lane 4).

### Primer Sequence

FLO1F1-
(Seq ID No: 23)
5′- GCA AGC TTA TGA CAA TGC CTC ATC GCT A- 3′
FLO1R1-
(Seq ID No: 24)
5′-GGA TCC CAG GAA TAA CGA CCG TTA ATA AAT T-3′

### PCR Conditions

98° C.—30 seconds
35 cycles
98° C.—10 seconds
67° C.—30 seconds
72° C.—2 minutes
Final—72° C.—10 minutes

### Mitochondrial DNA

Mitochondrial DNA was extracted from lager and ale strains and used for subsequent PCR analysis. Analysis of the S. cerevisiae COB gene allowed polymorphic regions to be identified within the gene, and primers to be designed to these regions to allow ale and lager yeast strains to be differentiated. Cytochrome b, encoded by the mitochondrial COB gene is the only mitochondrially-encoded subunit of the cytochrome bc1 complex. With reference to FIG. 3, the primers designed are illustrated and are shown to cross the gene inside and outside the exon regions.

A combination of primers designated F1 and R1 were used to differentiate lager and ale strains successfully.

The combination of primers F1 and R1 were used in a PCR amplification of the COB gene. The electrophoresis profile of this amplification is shown in FIG. 4, and clearly shows that the COB gene can be used to differentiate between lager and ale strains. Bands from lanes 2, 3, 4 and 5 were isolated and then purified using Qiaquick gel extraction kit (Qiagen). The DNA sequences of the fragments in the bands were confirmed.

### Primer Sequences

COBF1
(SEQ ID NO: 5)
5′- CAA ATG TGT ATT TAA GTT TAG TGA ATA GTT ATA-3′
COBR1
(SEQ ID NO: 6)
5′- CCT ATC ACA ATT GTC ACA TTG AGG-3′

### PCR Conditions

98° C.—30 seconds
35 cycles
98° C.—10 seconds
61° C.—30 second
72° C.—1 minute 30 seconds

By comparing the mitochondrial DNA of lager and ale yeast strains a different gene order was observed (FIG. 5). By using primers to these different genes the different gene order can be exploited to allow lager and ale yeast strains to be distinguished. The results are summarised in Table 3. If two primers are too far apart the PCR will not be successful, and no amplification product will be observed.

TABLE 3
Identification of primer combination to differentiate ale and
lager brewing yeast strains using flanking regions of mtDNA genes.

| | Bands in ale | Bands in | Control |
|---|---|---|---|
| Primer combination | strain | lager | bands |
| COX3F-RPM1R | Yes | No | |
| ATP6F-ATP9R | No | Yes | |
| VAR1F1-COX2R | Yes | No | |
| COBF-ATP9R | Yes | No | |
| ATP9R-VAR1F | Yes | Yes | Control |
| ATP8F-ATP6R | Yes | Yes | Control |

### Primers Used:

COBF
(SEQ ID NO: 25)
5′GTTTTATTCTATATCGGTAGAG-3′
RPM1R
(SEQ ID NO: 26)
5′GGCGGGCCGGACTATATTTATATATTTATTAA-3′
ATP8F
(SEQ ID NO: 27)
5′GGATATGTCTGGGCTATTTTAACAGC-3′
ATP9R
(SEQ ID NO: 28)
5′TCCTAATAAACCAATTGTTGAGATACCTGCTCC-3′
VAR1F1
(SEQ ID NO: 29)
5′GTTCACCGGATTGGTCCCGC-3′
COX2R
(SEQ ID NO: 30)
5′GTTAATTGTAATCTTAATAAATC-3′
COX3F
(SEQ ID NO: 31)
5′CAGCTGGACATCATGTTGGATATGAAACAAC-3′
ATP6F
(SEQ ID NO: 32)
5′GGATATGTCTGGGCTATTTTAACAGC-3′
ATP6R
(SEQ ID NO: 33)
5′GTCTAATCTCAAATTGATCTAATGGTGATG-3′

With reference to FIG. 5, using primers designed across the flanking regions of COB, RPM1 and ATP9 genes, it molecular makers to differentiate ale and lager strains can be identified. Oligonucleotides designed across flanking regions of ATP8 and ATP6 with ATP9 and VAR1 can be used as control markers within the brewing yeast strains.

### Genetic Instability

FIG. 10 demonstrates that the primer pair COBF2 and COBR1

| | |
|---|---|
| COBF2 | |
| 5′ATGGCATTTAGAAAATCAAATG-3′ | (SEQ ID No. 34) |
| COBR1 | |
| 5′CCTATCACAATTGTCACATTGAGG-3′ | (SEQ ID No. 6) |

directed to the mitochondrial COB gene can be used to show genetic instability in the petite mutant of UNYC2, as observed by a different electrophoretic profile in the petite mutant compared to the non-mutant parent. This difference in profile is indicative of rearrangement and instability in the mitochondrial genome.

The PCR conditions used were:

98° C.—30 seconds
35 cycles
98° C.—10 seconds
54° C.—30 second
72° C.—2 minutes
72° C.—10 minutes

### Further Examples

The following further examples illustrate some of the sequences used to differentiate ale and lager strains along with unique sequences to differentiate specific lager strains.

Results below were based on mitochondrial genome sequence of brewing yeast strains.

The lager brewing strain S. pastorianus Weihenstephan 34/70 (W34) was provided by Fachhochschule Weihenstephan, Freising, Germany. Nakao and colleagues published (Nakao et al., 2009. DNA Res. 2009 April; 16(2):115-29) the whole genome sequence of the Weihenstephan 34/70 (W34) sequence obtained from the same supplier. This was published in DDBJ/EMBL/GenBank under the project accession ABPO00000000. The accession number for the mitochondrial genome is EU852811.

In this example, the W34 complete mitochondrial sequence was analysed independently using two different techniques: Sanger sequencing on ABI 3730 x1 and de novo sequencing with Roche GS FLX titanium chemistry. Furthermore strain LBY11, a lager brewing yeast strain widely used in brewing industry was sequenced using GS-FLX titanium chemistry.

Strains sequenced by University of Nottingham are referred to as W34UON, LBY11UON and the sequence published by the Japanese group as W34PUB (Nakao and collegues project accession ABPO00000000).

New primer sequences were developed based on mitochondrial DNA sequences in order to differentiate ale and lager strains using standard and Real-Time PCR. Subsequently, specific mitochondrial genomic sequence regions have been identified in order to differentiate key lager strains.

### Example A

### Yeast Strain Differentiation

### 1) Ale and Lager Differentiation Using Standard PCR (See FIGS. 12A and 12B).

### Primer Pair 1

Primers

| | |
|---|---|
| 10F1- 5′-agctatttttagtggtatgg3′ | (SEQ ID NO: 35) |
| 10R1- 5′-tttatttacagttcatcctg-3′ | (SEQ ID NO: 36) |

PCR conditions—98° C. for 2 min; 35 cycles of following conditions; 98° C. for 10 sec; 60° C. for 30 sec; 72° C. for 2 mins followed by 72° C. for 10 mins.

### Sequence Information

S. pastorianus W34UON sequence of gene COX1 (encodes subunit I of cytochrome c oxidase (46535-49348)) (SEQ ID NO: 37) corresponding to the band present in FIG. 12A:

agctatttttagtggtatggcaggaacagcaatgtctttaatcattag
attagaattagctgcacctggttcacaatatttacaaggaaatgctca
gttatttaatgttttagtagttggtcatgctgtattaatgattttctg
tgcgccattttgcttaatttatcactgtattgaagtgttaattgataa
acatatctctgtttattcaataaatgaaaactttaccgtatcattttg
gttctgattcttagtagtaacatacatagaatttagatacgtaaacca
tatggcttactcagttggggccaactcaacggggacaatagcatgcca
taaaagcgctggagtaaaacagccagtgcaaggtaagaactgttcgat
ggctaggttaacgaactccttacaagaatgtttagggttctcattaac
tccttcccactcggggattgtggttcatgcttgtgtattggaagaaga
ggtacacgagttaaccaaatatgaatcattaactttaagtaaaagttg
acattcggagagctgtacgagttcaaatggtaaattaagaaatatggg
attgtccgaaaggggaaactctggggataacggagtcttcatagtacc
caaatttaatttaaataaagtgagatattttagtactttatctagatt
aaatgtaaggaaggaagacagtttaacgtatttaacaaagataaatac
tacggatttttccgagttaaataaattaatagaaaataattataataa
tcctgaaaacattaatactagaattttaaaattaatgtcagatattag
attgttattaattgcttataataaaattaaaagtaagaaaggtaacat
atctaaaggttctaataatattaccttagatggaattaatatttcata
tttaaataaattatctaaagatattaatactaatatgtttaaattttc
tcctgttagaagagttgaaattcctaaaacatctggaggatttagacc
tttaagtgttggtaatcctagagaaaaaattgtacaagaaagtatgag
aataattttagaaattatttataataatagtttttctaattattcaca
tgggtttagaccaaacttatcttgtttaacagctattattcattgtaa
aaattatatgcaacactgtaattgatttattaaggtagacttaaataa
atgttttgatacaattccacataatatgttaattaatgtattaaatga
gagaatcaaagataaaggtttcattgatttattatataaattattaag
agctggatatgttgataaacataataattatcatcatacaactttagg
aattcttcaaggtagtgttgtcagtcctattttatgtaatattttctt
agataaattagataaatatttagaaaataagtttgagaatgaattcaa
tattggatctatgtctaatagaagtagaaatccaatttataatgattt
atcatctaaaattagaagatgtaaattattatctgataaattaaaatt
gattagattaagagaccattaccaaagaaatttgggatctgataaaag
ctttaaaagagettattttgttagatatgctgatgatattatcattgg
tgtaatgggttctcatgatgattgtaaaaatattttaaacgatataaa
taatttcttaacagaaaatttaggtatgtctattaatatagataaatc
cattattaaacattctaaagaaggagttagttttttagggtatgatgt
aaaagttacaccttgagaaataagaccttatagaatgattaaaaaagg
tgataaatttattagggttagacatcatactagtttagttgttaatgc
ccctatcagaagtattgtaataaaattaaataaaaatggttattgttc
tcatggaatagttggaaaacccataggggttggaagattaattcatga
agaaatgaaaaccattttaatgcattatttagctgttggtagaggtat
tataaattattatagattagctaccaattttactacattaagaggtag
aattacatacattttattttattcatgttgtttaacgttagcaagaaa
atttaaattaaatactgttaagaaagttattttaaaattcggtaaagt
attgaccgatcctaattcaaaagtaagttttggtattgatgattttaa
aattagacataaaataaataaaactgattctaattatactcctgatga
aattttagatagatttaaatatatgttacctagatctttatcattatt
tagtggtatttgtcaagtttgtggttctaaacaaaatttagaagtaca
tcatgtgaaaatattaaataatgctgccaataaaatcaaaaatgatta
tttattaggtagaatgattaagataaatagaaaacaaattactatctg
taaaacatgtcattataaaattcatcaaggtaaatataatggtccagg
tttataataattattttaactattaactacgcgttaaatggagagccg
tatgatatgaaagtatcacgtacggttcggagagggctctcttatatg
attgttaatacaatcagataggtttgctactctactettagtaatgcc
agctttaattgggggttttggtaattatttattacccttaatgattgg
tgctacagatacagcatttccaagaattaataatattgcattttgagt
attacctatgggattagtatgtttagttacatcaactttagtagaatc
aggtgctggtacaggatgaactgtaaataaa

### Primer Pair 2 (See FIGS. 13A and 13B)

| | | |
|---|---|---|
| COX1F1 | 5′-ctacagatacagcatttcca-3′ | (SEQ ID NO: 38) |
| 10R5 | 5′-cgctgtaatgaaaattgatc-3′ | (SEQ ID NO: 39) |

PCR conditions—94° C. for 2 min; 35 cycles of following conditions; 94° C. for 10 sec; 58.5° C. for 30 sec; 68° C. for 2 mins followed by 68° C. for 7 mins.

### Sequence Information

S. pastorianus W34UON sequence of gene COX1 (49224-50208) (SEQ ID NO: 40) corresponding to the band present in FIG. 13A:

ctacagatacagcatttccaagaattaataatattgcattttgagtat
tacctatgggattagtatgtttagttacatcaactttagtagaatcag
gtgctggtacaggatgaactgtaaataaaaaggatatgcagttttaaa
atatcatttaatgcataaaataccttatatataataaatattatatat
aattattaaacatacttaatatatatatatatattaataataatatat
taagttataatgtttaataatataggttaatatgcaaatatttataat
attaataaatatttcagagactaaatatgatataatatattaatatat
taattatctttttataaaaaaaaactattctcattgtaccgaccgtta
gatacgacgatcgacactattaaatatgatatttataattaataatta
ttttctttataaaatcaatttgatgaataatataattagatttaatta
catttatgaaatatttataaatataatttaattaatatataatttttt
cccgtggatcaaccctattaacaactgggttgtaatttgggggtaata
aatattattatattatttttttattataaaaataaagtatataaatac
tttatattactataataatttttttatatatttataatataattaatt
tatattaatatattaaagacatagtccgaacaatatagtaatatattg
agatatagatattatatatatatttatataaacaattataataattaa
atattatttaattattaatttatgatatccaccattatcatctattca
ggcacattcaggacctagtgtagatttagcaatttttgcattacattt
aacatcaatttcatcattattaggtgctattaatttcattgtaacaac
attaaatatgagaacaaatggtatgacaatgcataaattaccattatt
tgtatgatcaattttcattacagcg

### 2) Ale and Lager Differentiation Using Real-Time PCR (See FIGS. 14A-C) Targeting the COB Gene (Cytochrome b)

### Primers

(SEQ ID NO: 41)
QF2 5′-aatggttattatgcatatgatggcattac-3′
(SEQ ID NO: 42)
QR3 5′-cctgtaatacctaatggattagatg-3′
(SEQ ID NO: 43)
QR4 5′-caggatgacctaaagtatttggtg-3′

PCR conditions—95° C. for 30 S, 40 cycles of following conditions, 95° C. for 3 S; 59° C. 30 s followed by 95° C. 15 s, 59° C. 1 m and 95° C. 15 s to produce melt curve.

S. pastorianus Sequence

### Product 1 for QF2 and QR3 (41177 to 41242) is Generated in Ale and Lager Strains:

(SEQ ID NO: 44)
aatggttattatgcatatgatggcattacatattcatggttcatctaa
tccattaggtattacagg

### Product 1 for QF2 and QR4 (41177 to 41364) Only Generated in Lager Strains:

(SEQ ID NO: 45)
aatggttattatgcatatgatggcattacatattcatggttcatctaa
tccattaggtattacaggtaatttagatagaattccaatgcattcata
tttcgtatttaaagatttagtaactgtatttttatttatgttagtatt
agcattatttgtattttattcaccaaatactttaggtcatcctg

### Target Sites for Primer Design

### 1. COB and COX1 Genes as Target Sites

Target sites for PCR differentiations using COB and COX1 gene sequences in W34UON have been identified in exons compared with S. cerevisiae (FIGS. 15 and 16). These target sites were utilised to develop primers in FIGS. 12A and 13A. Intron regions were also examined and potential target sites for PCR differentiation were identified. These target sites are important regions for the development of primers.

### 2. Comparative Genome Sequence Differences Between Strains W34PUB (Nakao et al., 2009. DNA Res. 2009 April; 16(2):115-29) and W34UON

W34PUB and W34UON sequences were analysed and the following sequence differences were identified which could be used to differentiate the two W34 strains using Real-Time PCR (FIG. 17). FIG. 17 regions A and B correspond to the genome regions of 16754 to 16855 and 16882 to 16914, respectively which both belong to 21S rRNA gene.

FIG. 17 region C corresponds to the genome region of 23896 to 23937 which is part of the COX2 gene. These nucleotide sequence changes do not alter amino acid sequence. However the differences identified in W34PUB when compared to W34UON sequence are also present in the closely related strain, LBY11UON.

### 3. Comparative Genome Sequence Differences Between W34PUB (Nakao et al., 2009. DNA Res. 2009 April; 16(2):115-29), W34UON and LBY11UON

LBY11UON is a lager brewing yeast strain widely used in beer fermentation in the UK. Two sequence differences have been identified in LBY11UON compared to W34PUB and W34UON (FIG. 18). In both cases these differences occur in repetitive sequences. The difference shown in FIG. 18 region D occurs 110 bp downstream of 21S rRNA gene. The sequence of FIG. 18 region E occurs in a non-coding region between ATP6 (mitochondrially encoded subunit A of the F0 sector of mitochondrial F1F0 ATP synthase, 56472-57251) and ATP9 (mitochondrially encoded F0-ATP synthase subunit C, 65894-66124).

### Example B

### Identification of Genetic Instability in Brewery Fermentation

Brewers re-use (re-pitch) their yeast until its genetic stability, viability, vitality or fermentation performance deteriorates. Due to stresses from successive fermentations and yeast handling the incidence of genetic drift and mutations is high (Morrison K B, Suggett A (1983). J Inst Brew 89: 141-142; Powell C D, et al. (2000). Lett Appl Microbiol 31(1): 46-51; Sato M, et al. (2001). J Am Soc Brew Chem 59(3): 130-134; Silhankova L, et al (1970). J Inst Brew 76: 280-288; Watari J, et al. (1999). Euro Brew Cony Mono 28: 148-159). The formation of petite mutants, which result from the loss of mitochondrial DNA (mtDNA) integrity, is one of the key indicators of genetic instability. Petite mutations are known to negatively affect the fermentation performance and beer flavour profiles (Smart K A (2007). Yeast 24:993-1013).

Mutations in mtDNA may result in the formation of two forms of petite mutant: rho⁻ mutants, in which mtDNA is present with specific genes or sequences deleted and the remaining sequences amplified (Bernardi G, et al. (1979). In Biochemistry and Genetics of Yeasts, Bacila M, Horecker B L, Stoppani A O M (eds), pp 241-254. Academic Press; Borst P & Grivell L A (1978). Cell 15(3): 705-723; Bos J L, et al. (1980). Cell 20(1): 207-214; Heyting C, et al. (1979). Mol Gen Genet 168(3): 231-246; Piskur J, et al. (1998). Int J Syst Bacteriol 48 Pt 3: 1015-1024); and rho⁰ mutants, in which no mtDNA remains (Heidenreich E & Wintersberger U (1997). Curr Genet 31(5): 408-413; Rickwood R, et al. (1991). In Yeast a practical approach, Campbell I, Duffus J H (eds), p 185. Oxford: IRL Press).

Ale (Silhankova L, et al (1970). J Inst Brew 76: 280-288) and lager (Gibson B R, et al. (2006). Cerevisia 31(1): 25-36; Jenkins C L, et al. (2009). J Am Soc Brew Chem 67(2):72-80; Martin V, et al. (2003) In Brewing Yeast Fermentation Performance, 2nd Edition, Smart K A (ed), pp 61-73. Oxford: Blackwell Science) yeast exhibit different susceptibilities to petite formation, potentially as a consequence of their capacity to elicit stress and repair responses to the conditions which favour petite formation. Since all mtDNA must be damaged for a petite mutant to be formed, susceptibility of a given strain to form petites may also be a function of the mtDNA copy number (typically 20-50 in Saccharomyces species).

1. Development of mtDNA Restriction Digest Techniques to Differentiate Lager Yeast Strains from their Petite Mutant Strains

Typically, mtDNA has a low GC content when compared with the nuclear DNA of the same species (de Zamaroczy M & Bernardi G (1986). Gene 47(2-3): 155-177). Thus, differences in mtDNA restriction patterns can be revealed by digesting total genomic DNA with endonucleases which recognise GC-rich nucleotide regions. In this way, the nuclear DNA is cleaved into many small fragments, allowing higher molecular weight mtDNA bands to appear (Typas M A, et al. (1992). FEMS Microbiology Letters 95(2-3): 157-162). Total genomic DNA was extracted from LBY11UON and petites isolated from the strain. RFLP (Restriction Fragment Length Polymorphism) pattern was produced by digesting DNA with the endonuclease Haan (GG^{↓}CC) (see FIG. 19).

This shows that RFLP patterns can be used to determine the stability of the yeast strain by comparing the RFLP pattern with a known conserved RFLP pattern of a stable yeast.

TABLE 4
Example of restriction fragment lengths expected for a known
conserved RFLP pattern in a stable LBY11UON strain when
cut by either HaeIII or HinfI.

| | |
|---|---|
| Size bp | Position |

HaeIII RFLP pattern

| | |
|---|---|
| 5567: | LBY11UON: HaeIII(23913)-HaeIII(29480) |
| 5303: | LBY11UON: HaeIII(37477)-HaeIII(42780) |
| 4471: | LBY11UON: HaeIII(51084)-HaeIII(55555) |
| 4248: | LBY11UON: HaeIII(46836)-HaeIII(51084) |
| 3008: | LBY11UON: HaeIII(8177)-HaeIII(11185) |
| 2812: | LBY11UON: HaeIII(1798)-HaeIII(4610) |
| 2539: | LBY11UON: HaeIII(61216)-HaeIII(63755) |
| 2492: | LBY11UON: HaeIII(67188)-HaeIII(69680) |
| 2437: | LBY11UON: HaeIII(34468)-HaeIII(36905) |
| 2195: | LBY11UON: HaeIII(32005)-HaeIII(34200) |
| 2052: | LBY11UON: HaeIII(44784)-HaeIII(46836) |
| 1916: | LBY11UON: HaeIII(13313)-HaeIII(15229) |
| 1744: | LBY11UON: HaeIII(57309)-HaeIII(59053) |
| 1545: | LBY11UON: HaeIII(22339)-HaeIII(23884) |
| 1542: | LBY11UON: HaeIII(55767)-HaeIII(57309) |
| 1495: | LBY11UON: HaeIII(15693)-HaeIII(17188) |
| 1414: | LBY11UON: HaeIII(17294)-HaeIII(18708) |
| 1396: | LBY11UON: HaeIII(20930)-HaeIII(22326) |
| 1194: | LBY11UON: HaeIII(19228)-HaeIII(20422) |
| 1063: | LBY11UON: HaeIII(30314)-HaeIII(31377) |
| 1060: | LBY11UON: HaeIII(60156)-HaeIII(61216) |
| 1004: | LBY11UON: HaeIII(64468)-HaeIII(65472) |
| 849: | LBY11UON: HaeIII(59307)-HaeIII(60156) |
| 765: | LBY11UON: HaeIII(65485)-HaeIII(66250) |
| 760: | LBY11UON: HaeIII(87)-HaeIII(847) |
| 733: | LBY11UON: HaeIII(7378)-HaeIII(8111) |
| 720: | LBY11UON: HaeIII(6554)-HaeIII(7274) |
| 713: | LBY11UON: HaeIII(63755)-HaeIII(64468) |
| 686: | LBY11UON: HaeIII(5397)-HaeIII(6083) |
| 674: | LBY11UON: HaeIII(66250)-HaeIII(66924) |
| 672: | LBY11UON: HaeIII(4725)-HaeIII(5397) |
| 589: | LBY11UON: HaeIII(847)-HaeIII(1436) |
| 587: | LBY11UON: HaeIII(29480)-HaeIII(30067) |
| 572: | LBY11UON: HaeIII(36905)-HaeIII(37477) |
| 564: | LBY11UON: HaeIII(43962)-HaeIII(44526) |
| 549: | LBY11UON: HaeIII(43413)-HaeIII(43962) |
| 539: | LBY11UON: HaeIII(11560)-HaeIII(12099) |
| 503: | LBY11UON: HaeIII(70077)-end(70579) |
| 498: | LBY11UON: HaeIII(20432)-HaeIII(20930) |
| 464: | LBY11UON: HaeIII(15229)-HaeIII(15693) |
| 460: | LBY11UON: HaeIII(12840)-HaeIII(13300) |
| 416: | LBY11UON: HaeIII(42780)-HaeIII(43196) |
| 393: | LBY11UON: HaeIII(31377)-HaeIII(31770) |
| 388: | LBY11UON: HaeIII(69680)-HaeIII(70068) |
| 382: | LBY11UON: HaeIII(12458)-HaeIII(12840) |
| 378: | LBY11UON: HaeIII(18718)-HaeIII(19096) |
| 375: | LBY11UON: HaeIII(11185)-HaeIII(11560) |
| 350: | LBY11UON: HaeIII(12108)-HaeIII(12458) |
| 312: | LBY11UON: HaeIII(6127)-HaeIII(6439) |
| 312: | LBY11UON: HaeIII(1486)-HaeIII(1798) |
| 254: | LBY11UON: HaeIII(59053)-HaeIII(59307) |
| 249: | LBY11UON: HaeIII(66924)-HaeIII(67173) |
| 248: | LBY11UON: HaeIII(44536)-HaeIII(44784) |
| 237: | LBY11UON: HaeIII(30067)-HaeIII(30304) |
| 218: | LBY11UON: HaeIII(31787)-HaeIII(32005) |
| 204: | LBY11UON: HaeIII(43209)-HaeIII(43413) |
| 165: | LBY11UON: HaeIII(55564)-HaeIII(55729) |
| 144: | LBY11UON: HaeIII(34200)-HaeIII(34344) |
| 124: | LBY11UON: HaeIII(19104)-HaeIII(19228) |
| 115: | LBY11UON: HaeIII(4610)-HaeIII(4725) |
| 106: | LBY11UON: HaeIII(17188)-HaeIII(17294) |
| 104: | LBY11UON: HaeIII(7274)-HaeIII(7378) |
| 89: | LBY11UON: HaeIII(34344)-HaeIII(34433) |
| 76: | LBY11UON: HaeIII(6478)-HaeIII(6554) |
| 62: | LBY11UON: HaeIII(25)-HaeIII(87) |
| 57: | LBY11UON: HaeIII(8120)-HaeIII(8177) |
| 39: | LBY11UON: HaeIII(6439)-HaeIII(6478) |
| 38: | LBY11UON: HaeIII(55729)-HaeIII(55767) |
| 35: | LBY11UON: HaeIII(34433)-HaeIII(34468) |
| 29: | LBY11UON: HaeIII(23884)-HaeIII(23913) |
| 26: | LBY11UON: HaeIII(6092)-HaeIII(6118) |
| 26: | LBY11UON: HaeIII(1451)-HaeIII(1477) |
| 17: | LBY11UON: HaeIII(31770)-HaeIII(31787) |
| 16: | LBY11UON: start(1)-HaeIII(17) |
| 15: | LBY11UON: HaeIII(67173)-HaeIII(67188) |
| 15: | LBY11UON: HaeIII(1436)-HaeIII(1451) |
| 13: | LBY11UON: HaeIII(65472)-HaeIII(65485) |
| 13: | LBY11UON: HaeIII(43196)-HaeIII(43209) |
| 13: | LBY11UON: HaeIII(22326)-HaeIII(22339) |
| 13: | LBY11UON: HaeIII(13300)-HaeIII(13313) |
| 10: | LBY11UON: HaeIII(44526)-HaeIII(44536) |
| 10: | LBY11UON: HaeIII(30304)-HaeIII(30314) |
| 10: | LBY11UON: HaeIII(20422)-HaeIII(20432) |
| 10: | LBY11UON: HaeIII(18708)-HaeIII(18718) |
| 9: | LBY11UON: HaeIII(70068)-HaeIII(70077) |
| 9: | LBY11UON: HaeIII(55555)-HaeIII(55564) |
| 9: | LBY11UON: HaeIII(12099)-HaeIII(12108) |
| 9: | LBY11UON: HaeIII(8111)-HaeIII(8120) |
| 9: | LBY11UON: HaeIII(6118)-HaeIII(6127) |
| 9: | LBY11UON: HaeIII(6083)-HaeIII(6092) |
| 9: | LBY11UON: HaeIII(1477)-HaeIII(1486) |
| 8: | LBY11UON: HaeIII(19096)-HaeIII(19104) |
| 8: | LBY11UON: HaeIII(17)-HaeIII(25) |

HinfI RFLP pattern

| | |
|---|---|
| 4889: | LBY11UON: HinfI(28164)-HinfI(33053) |
| 4189: | LBY11UON: HinfI(59343)-HinfI(63532) |
| 3027: | LBY11UON: HinfI(63532)-HinfI(66559) |
| 2861: | LBY11UON: HinfI(1468)-HinfI(4329) |
| 2528: | LBY11UON: HinfI(44246)-HinfI(46774) |
| 2411: | LBY11UON: HinfI(67757)-HinfI(70168) |
| 2230: | LBY11UON: HinfI(52604)-HinfI(54834) |
| 1944: | LBY11UON: HinfI(9378)-HinfI(11322) |
| 1924: | LBY11UON: HinfI(41129)-HinfI(43053) |
| 1804: | LBY11UON: HinfI(56808)-HinfI(58612) |
| 1663: | LBY11UON: HinfI(20682)-HinfI(22345) |
| 1622: | LBY11UON: HinfI(35412)-HinfI(37034) |
| 1606: | LBY11UON: HinfI(23631)-HinfI(25237) |
| 1537: | LBY11UON: HinfI(16688)-HinfI(18225) |
| 1497: | LBY11UON: HinfI(51029)-HinfI(52526) |
| 1454: | LBY11UON: HinfI(13319)-HinfI(14773) |
| 1346: | LBY11UON: HinfI(25237)-HinfI(26583) |
| 1298: | LBY11UON: HinfI(4676)-HinfI(5974) |
| 1228: | LBY11UON: HinfI(6133)-HinfI(7361) |
| 1220: | LBY11UON: HinfI(26944)-HinfI(28164) |
| 1198: | LBY11UON: HinfI(66559)-HinfI(67757) |
| 1196: | LBY11UON: HinfI(7361)-HinfI(8557) |
| 1167: | LBY11UON: HinfI(49308)-HinfI(50475) |
| 1128: | LBY11UON: HinfI(40001)-HinfI(41129) |
| 1110: | LBY11UON: HinfI(15412)-HinfI(16522) |
| 1101: | LBY11UON: start(1)-HinfI(1102) |
| 1021: | LBY11UON: HinfI(33053)-HinfI(34074) |
| 983: | LBY11UON: HinfI(47779)-HinfI(48762) |
| 943: | LBY11UON: HinfI(38234)-HinfI(39177) |
| 935: | LBY11UON: HinfI(22345)-HinfI(23280) |
| 837: | LBY11UON: HinfI(55971)-HinfI(56808) |
| 830: | LBY11UON: HinfI(12489)-HinfI(13319) |
| 792: | LBY11UON: HinfI(11322)-HinfI(12114) |
| 762: | LBY11UON: HinfI(34650)-HinfI(35412) |
| 738: | LBY11UON: HinfI(19175)-HinfI(19913) |
| 731: | LBY11UON: HinfI(58612)-HinfI(59343) |
| 712: | LBY11UON: HinfI(54834)-HinfI(55546) |
| 695: | LBY11UON: HinfI(37034)-HinfI(37729) |
| 642: | LBY11UON: HinfI(47137)-HinfI(47779) |
| 639: | LBY11UON: HinfI(14773)-HinfI(15412) |
| 593: | LBY11UON: HinfI(20035)-HinfI(20628) |
| 576: | LBY11UON: HinfI(34074)-HinfI(34650) |
| 566: | LBY11UON: HinfI(8812)-HinfI(9378) |
| 554: | LBY11UON: HinfI(50475)-HinfI(51029) |
| 546: | LBY11UON: HinfI(48762)-HinfI(49308) |
| 532: | LBY11UON: HinfI(18366)-HinfI(18898) |
| 505: | LBY11UON: HinfI(43741)-HinfI(44246) |
| 505: | LBY11UON: HinfI(37729)-HinfI(38234) |
| 435: | LBY11UON: HinfI(39566)-HinfI(40001) |
| 425: | LBY11UON: HinfI(55546)-HinfI(55971) |
| 412: | LBY11UON: HinfI(70168)-end(70579) |
| 389: | LBY11UON: HinfI(39177)-HinfI(39566) |
| 375: | LBY11UON: HinfI(12114)-HinfI(12489) |
| 366: | LBY11UON: HinfI(1102)-HinfI(1468) |
| 361: | LBY11UON: HinfI(26583)-HinfI(26944) |
| 351: | LBY11UON: HinfI(23280)-HinfI(23631) |
| 347: | LBY11UON: HinfI(4329)-HinfI(4676) |
| 315: | LBY11UON: HinfI(43187)-HinfI(43502) |
| 257: | LBY11UON: HinfI(46774)-HinfI(47031) |
| 255: | LBY11UON: HinfI(8557)-HinfI(8812) |
| 239: | LBY11UON: HinfI(43502)-HinfI(43741) |
| 212: | LBY11UON: HinfI(18963)-HinfI(19175) |
| 141: | LBY11UON: HinfI(18225)-HinfI(18366) |
| 134: | LBY11UON: HinfI(43053)-HinfI(43187) |
| 122: | LBY11UON: HinfI(19913)-HinfI(20035) |
| 106: | LBY11UON: HinfI(47031)-HinfI(47137) |
| 100: | LBY11UON: HinfI(5998)-HinfI(6098) |
| 90: | LBY11UON: HinfI(16522)-HinfI(16612) |
| 78: | LBY11UON: HinfI(52526)-HinfI(52604) |
| 76: | LBY11UON: HinfI(16612)-HinfI(16688) |
| 55: | LBY11UON: HinfI(18898)-HinfI(18953) |
| 54: | LBY11UON: HinfI(20628)-HinfI(20682) |
| 35: | LBY11UON: HinfI(6098)-HinfI(6133) |
| 24: | LBY11UON: HinfI(5974)-HinfI(5998) |
| 10: | LBY11UON: HinfI(18953)-HinfI(18963) |

2. Development of Techniques to Determine Relative mtDNA Copy Number

With reference to FIG. 21, a quantitative real-time PCR strategy was used to determine the relative amount of mtDNA and nuclear DNA. Pairs of primers designed for a nuclear DNA encoded gene, and a mitochondrially encoded gene were used in quantitative real-time PCR (Table 5). Products amplified by the primers were detected via fluorescence. As the amount of product increases, the fluorescence increases until a threshold fluorescence value (C_{T}) is exceeded. C_{T} was determined for both nuclear and mitochondrial genes. The difference (ΔC_{T}) between these C_{T} values is determined. The ΔC_{T} of the reference sample is subtracted from the experimental samples and the value obtained (ΔΔC_{T}) is then used to calculate the relative mtDNA copy number (RCN) of each sample, which is equal to 2^{-ΔΔ}^{_{CT}}. This technique was used to determine the relative mtDNA copy number of yeast samples collected during cropping of a full scale brewery fermentation vessel.

TABLE 5
Sequences of forward and reverse primers used
in real-time PCR reactions to determine
relative mtDNA copy number

| Gene | Component | Sequence |
|---|---|---|
| ACT1 | Forward Primer | 5′ CGCTCCTCGTGCTGTCTT 3′ |
| | (SEQ ID NO: 46) | |
| | Reverse Primer | 5′ TTGACCCATACCGACCATGA 3′ |
| | (SEQ ID NO: 47) | |
| COX2 | Forward Primer | 5′ GTAACAGCTGCAGATGTTATTCA 3′ |
| | (SEQ ID NO: 48) | |
| | Reverse Primer | 5′ CCATAGAAAACACCTTCTCTTTG 3 |
| | (SEQ ID NO: 49) | |

Design of Mitochondrial Primers for Real-Time PCR Determination of Relative mtDNA Copy Number

Primers for real-time PCR with the Applied Biosystems StepOne machine are optimally 20-mers with a melting temperature (Tₘ) of 58-60° C. and a GC content of 30-80%. In addition, at the 3′ end, the last 5 bases should contain no more than 3 G or C bases. For optimum efficiency amplicon length should be 50-150 bp and runs of identical bases must be avoided. A 585 bp fragment of the mitochondrial gene COX2 from several yeast strains has been sequenced (Rainieri S, et al. (2008). FEMS Yeast Res 8:586-596). Alignment of these sequences (FIG. 20) facilitated the design of primers for real-time PCR.

This technique was utilised to assess the relative mtDNA copy number of yeast samples collected during cropping of full scale brewery fermentation. The possible relationship between mtDNA copy number and petite frequency was then considered in individual crop samples. Analysis of these yeast samples indicated that low mtDNA copy number appears to correlate with higher frequency of petites.

Using this technique with DNA isolated from brewery petites, the petites samples had a relative copy number of zero when compared wild type LBY11UON strain (LBY11UON). This indicates that these petites have a deletion in the COX2 gene. Using this technique and data from petite mitochondrial genome sequence, the presence of petite mutations in brewery fermentation can be investigated (FIG. 21).

3. Sequencing of Mitochondrial Genome from LBY11UON Petite

A predominant petite mutant showing one particular RFLP profile (indicated by the arrows in FIGS. 19 and 22) was identified. Petites isolated from two separate brewery fermentations (8 months apart) exhibited the same predominant RFLP profiles with the enzymes HaeIII and HinfI (FIG. 22). Experiments with restriction enzymes DdeI and RsaI demonstrated similar results. Mitochondrial DNA from this petite was isolated and sequenced using GS-FLX standard technology. So far sequencing analysis has only generated 800 bp of data, encompassing ATP6. This sequence is identical to LBY11UON (FIG. 23) and. This region is hypothesised to be amplified (present in multiple copies) in this particular petite.

1. A method of determining the strain or strains of yeast in a sample, comprising:
obtaining nucleic acid from yeast in the sample;
screening the nucleic acid for two or more target sequences, wherein one target sequence in the nucleic acid comprises all or part of the COB gene, or a flanking region associated with the COB gene; and
determining from the results of the screen the yeast strain or strains in the sample.

2. A method of determining the genetic stability of a yeast strain in a sample, comprising:
obtaining nucleic acid from the yeast in the sample;
screening the nucleic acid for two or more target sequences, wherein at least of the one target sequences in the nucleic acid comprises all or part of the COB gene, or a flanking region associated with the COB gene;
comprises all or part of a gene, or a flanking region associated with a gene, in the yeast mitochondrial DNA or all or part of a gene, or a flanking region associated with a gene, located in the subtelomeric region of a chromosome;
determining from the results of the screen if the yeast strain is genetically stable.

3. The method of claim 1, wherein the screening step of the invention is performed using PCR to amplify the two or more target sequences.

4. The method of claim 3 wherein the presence or absence of the target sequence in the nucleic acid sample is determined by detecting the presence or absence of an amplification product from the PCR reaction.

5. The method of any preceding claim wherein the method can be carried out in less than 24 hours.

6. The method of claim 2 wherein the sample is obtained before, during or after a fermentation process.

7. The method of claim 2 wherein the method is performed on a sample obtained from a brewing process.

8. The method of any preceding claim wherein the sample is a liquid, slurry or solid.

9. The method of claim 2 wherein the target sequence further comprises at least part of a gene selected from the group comprising COX1, COX2, COX3, ATP8, ATP6, ATP9, VAR1, RPM1 the TIR genes, the DAN genes, the FLO genes, the ECM genes, the BUD genes, the KEL genes, the MNT genes, the SED genes, the MEL genes, SUC genes, the ATF genes, the GST genes, the GAL genes, MAL1-4, CYC1, CYC2, CYC3, CBP1, CBP2, CBP3, CBP4, and CBT1, and/or any non-coding sequences flanking or separating these genes, or combinations thereof.

10. The method of any preceding claim wherein at least one of the target sequences comprises all or part of at least one non-mitochondrial gene, or the flanking region associated with at least one non-mitochondrial gene.

11. The method of claim 10 wherein the non-mitochondrial gene, and/or flanking sequence thereof, is a gene which encodes a protein associated with the yeast cell wall and/or a protein which is involved in sugar metabolism.

12. The method of claim 10 wherein the target sequence of DNA comprises all or at least part of one or more of the yeast genes selected from the group comprising the TIR genes, the DAN genes, the FLO genes, the ECM genes, the BUD genes, the KEL genes, the MNT genes, the SED genes, the MEL genes, SUC genes, the ATF genes, the GST genes, the GAL genes, MAL1-4, CYC1, CYC2, CYC3, CBP1, CBP2, CBP3, CBP4, and CBT1 and/or a flanking region thereof.

13. The method of any preceding claim, as it depends on claim 2, wherein one or more target sequences comprise at least a part of a gene selected from the group comprising COB, COX1, COX2, COX3, ATP8, ATP6, ATP9, VAR1, RPM1, ECM34, SUC1, SUC3, SUC4, SUC5, SUC7, MAL1, MAL2, MAL3, MAL4, MEL2, MEL3, MEL4, MEL5, MEL6, MEL7, MEL8, MEL9 and MEL10 and/or any flanking regions of these genes, or combinations thereof.

14. The method of any preceding claim wherein one or more oligonucleotide primers or probes complementary or reverse complementary to the target sequence are used to detect the target sequence.

15. The method of claim 14 wherein one or more of the primers or probes comprises a sequence selected from the group comprising SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 38, 39, 41, 42 and 43, or combinations thereof, or a sequence with at least 80%, 85%, 90%, 95%, 98% or more sequence identity to a sequence with the sequence of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 38, 39, 41, 42 and 43.

16. A composition comprising one or more oligonucleotides having a sequence selected from the group comprising SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 38, 39, 41, 42, 43, 46, 47, 48, and 49 or a sequence with at least 80%, 85%, 90%, 95%, 98% or more sequence homology.

17. A kit for determining the strain or strains of yeast in a sample comprising two or more primers or probes directed to two or more target sequences in the yeast nucleic acid, wherein the target sequence comprises all or part of the COB gene, or a flanking region associated with the COB gene, and all or part of the TIR4, or a flanking region associated with the TIR4 gene.

18. A kit for determining the stability of a strain of yeast in a sample comprising two or more primers or probes directed to two or more target sequences in the yeast nucleic acid, wherein the target sequence comprises all or part of the COB gene, or a flanking region associated with the COB gene, and all or part of the COX2 gene, or a flanking region associated with the COX2 gene.

19. The kit according to claim 18, wherein the kit is used with PCR.

20. A kit according to claims 17 to 19 further comprising a PCR reagent.

21. A kit according to any of claims 17 to 20 comprising instructions to use kit, including the PCR conditions to use.

22. A kit according to any of claims 17 to 21 comprising details of the expected size of one or more amplification products.

23. A kit according to any of claims 17 to 21 comprising primers or probes directed to all or part of one or more of the following genes, or the flanking sequences thereof, COB, COX1, COX2, COX3, ATP8, ATP6, ATP9, VAR1 and RPM1 or combinations thereof.

24. A kit according to any of claims 17 to 23 comprising one or more primers or probes directed to all or at least part of one or more of the yeast genes selected from the group comprising the TIR genes, the DAN genes, the FLO genes, the ECM genes, the BUD genes, the KEL genes, the MNT genes, the SED genes, the MEL genes, SUC genes, the ATF genes, the GST genes, the GAL genes, MAL1-4, CYC1, CYC2, CYC3, CBP1, CBP2, CBP3, CBP4, and CBT1 and/or a flanking region thereof.

25. A kit according to any of claims 17 to 22 comprising one or more primers or probes selected from the group comprising SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 38, 39, 41, 42 and 43, or combinations thereof, or a sequence with at least 80%, 85%, 90%, 95%, 98% or more sequence identity to a sequence with the sequence of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 38, 39, 41, 42 and 43.

26. A method of analysing a yeast-containing sample comprising using a probe or primer in said analysis; wherein the probe or primer is capable of hybridising to all or part of the COB gene, or a flanking region associated with the COB gene, and all or part of the TIR4 and/or COX2 gene, or a flanking region associated with the TIR4 and/or COX2 gene if said yeast is present and/or is stable in said sample.

27. A method according to claim 26, wherein the probe or primer is not capable of hybridising to mitochondrial DNA of a further yeast, if present, in said sample.

28. A method according to claim 1 or claim 2 wherein the given yeast is S. pastorianus or S. cerevisiae.

29. A method according to claim 2, wherein the further yeast is S. pastorianus or S. cerevisiae.

30. A method according to any of claims 1 to 15 and 26 to 29, or a composition according to claim 16, or kit according to claims 17 to 25, for use to determine whether or not a yeast containing sample contains an undesired yeast.

31. A method according to any of claims 1 to 15 and 26 to 30, or a composition according to claim 16, or kit according to claims 17 to 25, for use in checking the quality of a sample intended for use in subsequent fermentation involving yeast.

32. A method according to claim 31 comprising the step of performing fermentation using said yeast if the quality is acceptable or aborting fermentation if the quality is not acceptable.

33. A probe or primer suitable for use in a method according to any of claims 1 to 15 and 26 to 32.

34. The probe or primer of claim 33, wherein the probe or primer preferentially hybridises to mtDNA of S. pastorianus, or preferentially hybridises to mtDNA of S. cerevisiae.

35. The probe or primer of claim 33 or claim 34, wherein the probe or primer preferentially hybridises to any of the genes selected from COB, COX1, COX2, COX3, ATP8, ATP6, ATP9, VAR1 and RPM1.

36. The probe or primer of any of claims 33 to 35, wherein the probe or primer preferentially hybridises to any of the sequences selected from the group comprising SEQ ID NO: 37, 40, 44, 45, 50, 51, 54, 55, 58, 59, 60, 61, 62, 63, 64, 65, 66, 67, 68, 69, 70, 71, and 72, or complements thereof.

37. The probe or primer of claims 33 to 35, wherein the probe or primer preferentially hybridises to SEQ ID NO: 50 or SEQ ID NO: 54, or complements thereof, and optionally does not hybridise to SEQ ID NO: 51 or SEQ ID NO: 55, or complements thereof or wherein the probe or primer preferentially hybridises to SEQ ID NO: 51 or SEQ ID NO: 55, or complements thereof, and optionally does not hybridise to SEQ ID NO: 50 or SEQ ID NO: 54, or complements thereof.

38. The probe or primer of claims 33 to 35, wherein the probe or primer preferentially hybridises to any of SEQ ID NOS: 58, 59 or 60, or complements thereof, and optionally does not hybridise to any of SEQ ID NOS: 61, 62, 63, 64, 65, or 66, or complements thereof, or wherein the probe or primer preferentially hybridises to any of SEQ ID NOS: 61, 62, 63, 64, 65, or 66, or complements thereof, and optionally does not hybridise to any of SEQ ID NOS: 58, 59 or 60, or complements thereof.

39. The probe or primer of claims 33 to 35, wherein the probe or primer preferentially hybridises to any of SEQ ID NOS: 67 or 68, or complements thereof, and optionally does not hybridise to any of SEQ ID NOS: 69, 70, 71, or 72, or complements thereof, or wherein the probe or primer preferentially hybridises to any of SEQ ID NOS: 69, 70, 71, or 72, or complements thereof, and optionally does not hybridise to any of SEQ ID NOS: 67 or 68, or complements thereof.

40. The method of claim 2, further comprising the use of Real-Time PCR (RT-PCR) to determine relative mtDNA copy number of a gene in the yeast.

41. The method of claim 2, further comprising screening the yeast by digestion of the yeast mtDNA with a restriction enzyme, which specifically cuts the nucleic acid between a guanine nucleotide and a cytosine nucleotide (ĜAC) to provide an RFLP pattern (Restriction Fragment Length Polymorphism), wherein the RFLP pattern of the yeast nucleic acid is compared to a known conserved RFLP pattern from a yeast that is not unstable, and wherein the observation of a significant difference in RFLP pattern indicates an unstable yeast strain.

42. A method according to any of claims 1 to 15, 26 to 32, 39 and 40, or a composition according to claim 16, or a kit according to any of claims 16 to 25, or a probe or primer according to any of claims 33 to 38, for use in brewing.

43. The method of claim 1 further comprising differentiating between ale and lager yeast strains.

44. The method of claim 43 further comprising differentiating between different lager strains.

45. The method of claim 1, wherein a second target sequence comprises all or part of the TIR4 gene, or a flanking region associated with the TIR4 gene.

46. The method of claim 45, wherein the method further comprises differentiating between different ale strains.

47. The method of claim 1, wherein the screening step comprises using PCR to amplify the two or more target sequences.

48. The method of claim 1, wherein the sample is obtained before, during, or after a fermentation process.

49. The method of claim 1, wherein the method is performed on a sample obtained from a brewing process.

50. The method of claim 1, wherein the target sequence further comprises at least part of a gene selected from the group comprising COX1, COX2, COX3, ATP8, ATP6, ATP9, VAR1, RPM1 the TIR genes, the DAN genes, the FLO genes, the ECM genes, the BUD genes, the KEL genes, the MNT genes, the SED genes, the MEL genes, SUC genes, the ATF genes, the GST genes, the GAL genes, MAL1-4, CYC1, CYC2, CYC3, CBP1, CBP2, CBP3, CBP4, and CBT1 and/or any non-coding sequences flanking or separating these genes, or combinations thereof.

51. The method of claim 2, wherein a second target sequence comprises all or part of the COX2 gene, or a flanking region associated with the COX2 gene.

52. The method of claim 2, wherein the yeast is an ale or lager yeast.

53. The kit according to claim 17, wherein the kit is used with PCR.
